(19) United States
(12) Patent Application Publication
(10) Pub. No.: US 2026/0011417 A1
(43) Pub. Date: Jan. 8, 2026

(54) SYSTEMS AND METHODS FOR HEALTH INFORMATION VISUALIZATION AND MANAGEMENT

(71) Applicant: NERv Technology Inc., Kitchener (CA)

(72) Inventors: Khaled Berry, Kitchener (CA); Abdulwadood AI-Ali, Kitchener (CA); Osama Awara, Kitchener (CA); Nour Hesham Atif Ahmed Helwa, Kitchener (CA); Abdallah Hassen El-Falou, Kitchener (CA); Ahmed Elsebay, Kitchener (CA); Manaswi Sharma, Kitchener (CA); Finnegan O'Hara, Waterloo (CA); Mazhar Shahen, Waterloo (CA)

(21) Appl. No.: 19/259,927

(22) Filed: Jul. 3, 2025

Related U.S. Application Data
(60) Provisional application No. 63/667,377, filed on Jul. 3, 2024.

(51) Int. Cl. G16H 10/60

(52) U.S. Cl. CPC G16H 10/60

(57) ABSTRACT

Systems and methods are disclosed herein for visualizing and managing health information data, the system comprising: one or more subsystems adapted to receive health information data of one or more patients; a display device configured to display a viewing framework; a computing system communicatively coupled to the one or more subsystems, the display device, a memory, and a processor, wherein the processor comprises program instructions that, when executed, causes the processor to perform operations comprising: receiving the health information data; determining clinical context information of the health information data to generate contextualized data; converting the contextualized data into usable data using trained machine learning models; detecting viewing parameters; determining the viewing framework based on the usable data and viewing parameters; and sending the viewing framework to the display device automatically.

### FIELD OF THE INVENTION

The present disclosure generally relates to systems and methods for managing healthcare information, in particular, systems and methods for processing and visualizing patient and/or medical device data from disparate healthcare information systems.

### BACKGROUND

Currently, patient care is fragmented into many different, inefficient systems. Nurses and clinicians often face significant time constraints, juggling multiple responsibilities and working tirelessly to ensure optimal patient outcomes. They are also under pressure to complete their current tasks while considering upcoming ones, making it challenging to maintain a steady workflow. This inefficiency leads to cognitive overload, delays in patient care, and potential errors at the expense of the health of patients.

Continuous advancements in medical technology, which include patient monitoring devices, require healthcare professionals to stay updated with the latest protocols on how to operate them. Patient monitoring devices may also be required to be regularly calibrated to ensure that accurate measurements are obtained. In some cases, patient monitoring devices must be calibrated every 24 hours to ensure optimal performance, adding an additional layer of complexity to the demanding workload of healthcare providers.

Although inventors of the present application have developed a computer-implemented AI-driven risk assessment method for patients, there do not exist easily understandable display methods such that healthcare professionals can interpret and act upon the data that is being measured, without needing to be an expert in AI models.

Further, there exist no methods for healthcare professionals to view multiple patients' conditions and/or statuses at the same time. In terms of the available data to healthcare professionals, there exists no means of consolidating and converting all of the different forms of patient data, structured and unstructured, hospital data, and literature, that can render the information useful and interpretable. The lack of integrated systems means that critical patient information can be scattered across different platforms, requiring additional time and effort to manage, consolidate, analyze, and interpret patient information.

Furthermore, doctors are generally cut off from viewing data relating to patients at other hospitals, and their currently exists no secure method of sharing patient data between hospitals in a secure and private manner that enables cross-hospital collaboration and allows healthcare professionals to observe cases that are similar to their own and may provide valuable insights in the development of their patient's care.

It is an object of this disclosure to provide systems and methods for seamlessly integrating and visualizing patient conditions and/or calibration data, that mitigates one or more of the above problems.

### BRIEF SUMMARY

The following presents a simplified summary of the general inventive concept(s) described herein to provide a basic understanding of some aspects of the disclosure. This summary is not an extensive overview of the disclosure. It is not intended to restrict key or critical elements of embodiments of the disclosure or to delineate their scope beyond that which is explicitly or implicitly described by the following description and claims.

The disclosure teaches systems and methods for seamlessly integrating and visualizing patient health information data, providing a comprehensive solution for healthcare professionals to receive a clear and concise overview of their patients' conditions. The object of the invention ultimately improves clinical workflow efficiency and enhances the reliability of healthcare delivery.

In accordance to an aspect, there is provided a system for visualizing and managing health information data, the system comprising: one or more subsystems adapted to receive health information data of one or more patients, wherein the one or more subsystems comprise one or more medical devices, electronic health records and inputs of a user; a display device configured to display a viewing framework; a computing system communicatively coupled to the one or more subsystems, the display device, a memory, and a processor, wherein the processor comprises program instructions that, when executed, causes the processor to perform operations comprising: receiving the health information data from the one or more subsystems; determining clinical context information of the health information data to generate contextualized data; converting the contextualized data into usable data using trained machine learning models; detecting viewing parameters, wherein the viewing parameters comprises one or more of: a display device type, a user's credentials, and a location of the display device; determining the viewing framework based on the usable data and viewing parameters; and sending the viewing framework to the display device automatically.

In some embodiments, the health information data comprises one or more of patient demographics, patient statuses, physiological measurements, medical history, medications, clinical notes, billing data, hospital guidelines and clinical research literature.

In some embodiments, the processor further performs operations comprising: determining one or more risk assessments related to the one or more patients; sorting the one or more risk assessments based on a level of risk; and identifying an alert relating to changes in the one or more risk assessments.

In some embodiments, the viewing framework comprises at least a graphical representation of one or more patient parameters relating to: vitals, medications, radiology tests, pre-operative data, intra-operative data, post-operative data, and fluid data.

In some embodiments, the viewing framework further comprises one or more risk assessments and one or more alerts.

In some embodiments, the one or more risk assessments is based on one or more thresholds relating to a condition of the one or more patients.

In some embodiments, the viewing framework further comprises a report displaying data tailored to one or more of a patient subgroup, a healthcare professional, and a hospital department.

In some embodiments, the viewing framework further comprises displaying one or more prompts for measuring vitals and taking tests, wherein the one or more prompts are updated and displayed based on a condition of the one or more patients.

In some embodiments, the viewing parameters may further comprise an index surgery, operative details, custom thresholds and a circle of care.

In some embodiments, the viewing parameters are customized by the user.

In accordance with another aspect, there is provided a method for visualizing and managing health information data, the method comprising: receiving, at one or more subsystems, health information data of one or more patients, wherein the one or more subsystems comprise one or more medical devices, electronic health records and user inputs; receiving, at a processor, the health information data from the one or more subsystems; determining, at the processor, clinical context information of the health information data to generate contextualized data; converting, at the processor, the contextualized data into usable data using trained machine learning models; detecting, at the processor, viewing parameters, wherein the viewing parameters comprises one or more of: a display device type, a user's credentials, and a location of the display device; determining, at the processor, the viewing framework based on the usable data and viewing parameters; sending, at the processor, the viewing framework to the display device automatically; and displaying, at the display device, the viewing framework.

In some embodiments, the health information data comprises one or more of patient demographics, patient statuses, physiological measurements, medical history, medications, clinical notes, billing data, hospital guidelines and clinical research literature.

In some embodiments, the method further comprises: determining, at a processor, one or more risk assessments related to the one or more patients; sorting, at the processor, the one or more risk assessments based on a level of risk; and identifying, at the processor, an alert relating to changes in the one or more risk assessments.

In some embodiments, the viewing framework comprises at least a graphical representation of one or more patient parameters relating to: vitals, medications, radiology tests, pre-operative data, intra-operative data, post-operative data, and fluid data.

In some embodiments, the viewing framework further comprises one or more risk assessments and one or more alerts.

In some embodiments, the one or more risk assessments is based on one or more thresholds relating to a condition of the one or more patients.

In some embodiments, the viewing framework further comprises a report displaying data tailored to one or more of a patient subgroup, a healthcare professional, and a hospital department.

In some embodiments, the viewing framework further comprises displaying one or more prompts for measuring vitals and taking tests, wherein the one or more prompts are updated and displayed based on a condition of the one or more patients.

In some embodiments, the viewing parameters may further comprise an index surgery, operative details, custom thresholds and a circle of care.

In some embodiments, the viewing parameters are customized by the user.

In accordance with a further aspect, there is provided a non-transitory computer readable medium storing program instructions, that when executed, cause a processor to perform operations comprising: receiving the health information data from the one or more subsystems; determining clinical context information of the health information data to generate contextualized data; converting the contextualized data into usable data using trained machine learning models; detecting viewing parameters, wherein the viewing parameters comprises one or more of: a display device type, a user's credentials, and a location of the display device; determining the viewing framework based on the usable data and viewing parameters; and sending the viewing framework to the display device automatically.

The advantages and features of the present invention will become better understood with reference to the following more detailed description and claims taken in conjunction with the accompanying drawings in which like elements are identified with like symbols.

### BRIEF DESCRIPTION OF THE DRAWINGS

To easily identify the discussion of any particular element or act, the most significant digit or digits in a reference number refer to the figure number in which that element is first introduced.

FIG. 1 illustrates a schematic of an exemplary computer system in accordance with one embodiment.

FIG. 2 illustrates a schematic of an exemplary computer system in accordance with one embodiment.

FIG. 3A illustrates a depiction of a user interface in accordance with one embodiment.

FIG. 3B illustrates a depiction of a user interface in accordance with one embodiment.

FIG. 3C illustrates a depiction of a user interface in accordance with one embodiment.

FIG. 3D illustrates a depiction of a user interface in accordance with one embodiment.

FIG. 3E illustrates a depiction of a user interface in accordance with one embodiment.

FIG. 3F illustrates a depiction of a user interface in accordance with one embodiment.

FIG. 3G illustrates a multi-patient depiction of a user interface in accordance with one embodiment.

FIG. 3H illustrates a depiction of an HCP dashboard in accordance with one embodiment.

FIG. 3I illustrates a viewing framework in a preoperative mode in accordance with one embodiment.

FIG. 3J illustrates a list of previous medical problems and surgical history of a patient in a viewing framework in accordance with one embodiment.

FIG. 3K illustrates customizable operative details in a viewing framework in accordance with one embodiment.

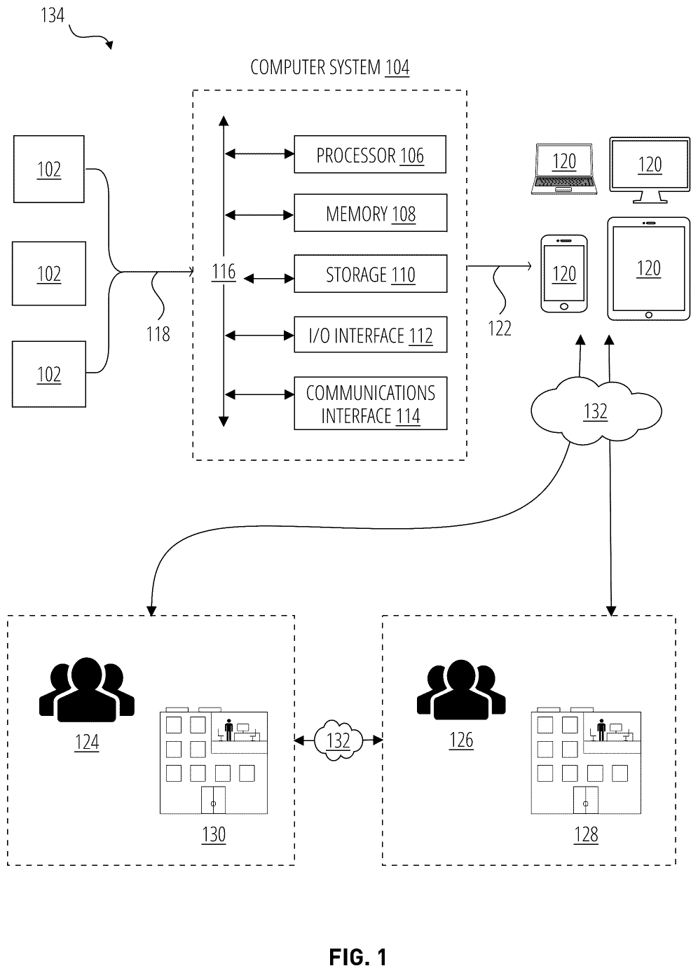

FIG. 1 illustrates customizable thresholds in a viewing framework in accordance with one embodiment.

FIG. 4 illustrates a depiction of a user interface on a mobile device in accordance with one embodiment.

FIG. 5 illustrates a depiction of a user interface having a 3D representation of a body in accordance with one embodiment.

FIG. 6 illustrates a flow chart depicting systems and methods for EHR integration in accordance with one embodiment.

FIG. 7A illustrates a user interface for tracking hospital metrics in accordance with one embodiment.

FIG. 7B illustrates a user interface for tracking doctor metrics in accordance with one embodiment.

FIG. 8 illustrates a diagram with a plurality of postoperative complications arranged according to different appropriate categories in accordance with one embodiment.

Elements in several drawings are illustrated for simplicity and clarity and have not necessarily been drawn to scale. For example, the dimensions of some of the elements in the figures may be emphasized relative to other elements for facilitating understanding of the various presently disclosed embodiments. Also, common, but well-understood elements that are useful or necessary in commercially feasible embodiments are often not depicted in order to facilitate a less obstructed view of these various embodiments of the present disclosure.

### DETAILED DESCRIPTION

Various implementations and aspects of the specification will be described with reference to the details discussed below. The following description and drawings are illustrative of the specification and are not to be construed as limiting the specification. Numerous specific details are described to provide a thorough understanding of various implementations of the present specification. However, in certain instances, well-known or conventional details are not described in order to provide a concise discussion of implementations of the present specification.

Furthermore, numerous specific details are set forth in order to provide a thorough understanding of the implementations described herein. However, it will be understood by those skilled in the relevant arts that the implementations described herein may be practiced without these specific details. In other instances, well-known methods, procedures and components have not been described in detail so as not to obscure the implementations described herein.

In this specification, elements may be described as “configured to” perform one or more functions or “configured for” such functions. In general, an element that is configured to perform or configured for performing a function is enabled to perform the function, or is suitable for performing the function, or is adapted to perform the function, or is operable to perform the function, or is otherwise capable of performing the function.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

### TERMS AND DEFINITIONS

Esper™: The Medical Device Management (MDM) service

HIPEC: Hyperthermic (or Heated) Intraperitoneal Chemotherapy is a surgical procedure for patients with abdominal cancers.

ADT (Admission, Discharge, and Transfer): ADT messages are used in healthcare settings to communicate patient admission, discharge, and transfer events within a facility.

EHR (Electronic Health Record): An EHR is a digital version of a patient's paper chart, providing real-time patient-centered records accessible to authorized healthcare providers.

FHIR (Fast Healthcare Interoperability Resources): FHIR is a standard describing data formats and elements (known as “resources”) and an API for exchanging EHR.

HCP (Healthcare Provider/Practitioner): An HCP is a professional who provides healthcare services to patients, including, but not limited to, doctors, nurses, and therapists.

HL7 v2 (Health Level Seven Version 2): HL7 v2 is a widely adopted standard for the exchange, integration, sharing, and retrieval of electronic health information.

HTTPS (HyperText Transfer Protocol Secure): HTTPS is an extension of HTTP that uses encryption via TLS to secure data exchanged between a user's browser and a web server.

PHI (Protected Health Information): PHI refers to any information in a medical record that can be used to identify an individual that was created, used, or disclosed during the provision of healthcare services.

TCP (Transmission Control Protocol): TCP is a standard that defines how to establish and maintain a network conversation through which application programs can exchange data.

TLS (Transport Layer Security): TLS is a cryptographic protocol designed to provide secure communication over a computer network, succeeding SSL.

VPN (Virtual Private Network): A VPN extends a private network across a public network, enabling users to send and receive data as if their computing devices were directly connected to the private network.

ASA classification or score (American Society of Anesthesiologists Physical Status Classification): A numeric score system to assess a patient's preoperative condition.

The following disclosure teaches systems and methods for digital patient care, data management, and healthcare displays. Following a surgical procedure, patients typically have a recovery period where they are monitored for the development of any complications. The current standard of care involves waiting for symptoms to appear and examining the patient before diagnosis and treatment. However, by the time a complication is diagnosed, the complication may have progressed to a more serious and critical stage. The longer detection takes, the worse the impact on the patient and HCP. The systems and methods further allow for early predictions of postoperative complications, ultimately allowing for early intervention as well. The disclosed systems and methods reduce the cost of treatment, improve quality of life, reduce morbidity and mortality, reduce the length of stay at hospitals, reduce readmission rates, and increase patient throughput (i.e., timely discharges to free up inpatient beds for new admissions).

The following disclosure further teaches systems and methods to streamline postoperative decision-making by integrating patient information and risk calculators into a single, centralized and intuitive interface by integrating EHRs, improving clinical efficiency and speed of decision making of HCPs. EHR data tends to be fragmented and is often presented across multiple notes, tables, workflows and screens, requiring relevant information to be filtered. The time-consuming process of gathering, navigating, cross-referencing and interpreting information from various sources increases the risk of overlooking critical details while reviewing patient charts that ultimately impacts patient outcomes. The platform automatically pre-populates patient data and provides risk stratification without requiring manual data entry at multiple stages, ensuring key details are not missed. By way of the disclosed systems and methods, HCPs can focus more on direct patient care rather than navigating EHRs and manually cross-referencing information, reducing cognitive burden.

The disclosed system comprises in accordance with different embodiments, a platform that is user-friendly and provides easily interpretable graphical representations of their trends in vital signs, lab test results, and fluid readings. The platform provides the user with risk scores against the various complications. The risk scores are dynamically computed based on standard clinical calculators, workflows and/or machine learning models. The system further provides contextualized alerts to prevent HCPs from inadvertently dismissing important alerts in bulk due to notification fatigue. For example, the contextualized alerts may allow for subtle trends in vital signs to be identified that may indicate a potential postoperative complication. These are all brought together and displayed in the main view of a dashboard providing HCPs with a holistic view of the status of their patients. In addition, the systems and methods herein provide a unified decision-making approach for junior HCPs to identify early and/or subtle signs of complications, especially when senior guidance is limited. The platform also allows junior HCPs to comply with hospital guidelines and communicate with senior HCPs regarding patient status, approval of correct tests or treatments, among others. This allows for better quality, consistency, and safety of postoperative care.

Existing risk calculators remain under-utilized due to the manual effort required to find, input data, and interpret results at multiple time points. This results in inconsistent decision-making and a reliance on subjective judgement rather than a standardized, data-driven process. This barrier to adoption also contributes to increasing variability in care, leading to higher risks of patients experiencing complications. The disclosed systems and methods may comprise one or more risk calculators which enable a more standardized, consistent, and objective approach to postoperative decision-making.

Systems and methods disclosed herein may be used in conjunction with devices, systems, and methods, previously disclosed by inventors of this application, including, but not limited to: PCT/CA2020/050395, PCT/CA2023/050896, PCT/CA2024/050131, PCT/CA2024/050135 U.S. patent Ser. No. 15/555,664, 16/804,897, U.S. patent application Ser. No. 18/456,096, 63/571,028, and 63/595,093, the contents of which are hereby incorporated by reference in their entirety. These include patient monitoring devices, display devices, and cloud services.

Devices and methods for carrying out the present disclosure are presented in terms of embodiments depicted within the FIGS. However, the present disclosure is not limited to the described embodiments, and a person skilled in the art will appreciate that many other embodiments of the present disclosure are possible without deviating from the basic concept of the present disclosure, and that any such work around will also fall under scope of this present disclosure. It is envisioned that other styles and configurations of the present disclosure can be easily incorporated into the teachings of the present disclosure, and the configurations shall be shown and described for purposes of clarity and disclosure and not by way of limitation of scope.

FIG. 1 is a simplified block diagram of a health information management system 134. According to one embodiment, the health information management system 134 develops a visualization framework for the visualization of a variety of data, measured by one or more subsystems 102. The one or more subsystems 102 measure or record data relating to one or more of: patient condition, patient treatment, hospital guidelines, literature (which may include, but is not limited to, clinical research literature having one or more guidelines for patient care), scheduling, HCP notes, and the like. The data obtained by the one or more subsystems 102 is sent to a computer system 104 via a subsystem information consolidation engine 118.

In the illustrated embodiment, the exemplary computer system 104 performs the methods as described or illustrated herein. In particular embodiments, one or more computer systems 104 perform one or more steps of one or more methods described or illustrated herein. In particular embodiments, one or more computer systems 104 provide functionality described or illustrated herein. In particular embodiments, software running on one or more computer systems 104 performs one or more steps of one or more methods described or illustrated herein or provides functionality described or illustrated herein. Particular embodiments include one or more portions of one or more computer systems 104. Herein, reference to a computer system may encompass a computing device, and vice versa, where appropriate. Moreover, reference to a computer system may encompass one or more computer systems, where appropriate.

This disclosure contemplates any suitable number of computer systems 104. This disclosure contemplates computer system 104 taking any suitable physical form. As example and not by way of limitation, computer system 104 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 104 may include one or more computer systems 104; be unitary or distributed; span multiple locations; span multiple machines; span multiple data centers; or reside in a cloud, which may include one or more cloud components in one or more networks. Where appropriate, one or more computer systems 104 may perform without substantial spatial or temporal limitation one or more steps of one or more methods described or illustrated herein. As an example, and not by way of limitation, one or more computer systems 104 may perform in real time or in batch mode one or more steps of one or more methods described or illustrated herein. One or more computer systems 104 may perform at different times or at different locations one or more steps of one or more methods described or illustrated herein, where appropriate.

In particular embodiments, computer system 104 includes a processor 106, memory 108, storage 110, an input/output (I/O) interface 112, a communication communications interface 114, and a bus 116. Although this disclosure describes and illustrates a particular computer system having a particular number of particular components in a particular arrangement, this disclosure contemplates any suitable computer system having any suitable number of any suitable components in any suitable arrangement.

In particular embodiments, processor 106 includes hardware for executing instructions, such as those making up a computer program. As an example, and not by way of limitation, to execute instructions, processor 106 may retrieve (or fetch) the instructions from an internal register, an internal cache, memory 108, or storage 110; decode and execute them; and then write one or more results to an internal register, an internal cache, memory 108, or storage 110. In particular embodiments, processor 106 may include one or more internal caches for data, instructions, or addresses. This disclosure contemplates processor 106 including any suitable number of internal caches, where appropriate. As an example, and not by way of limitation, processor 106 may include one or more instruction caches, one or more data caches, and one or more translation lookaside buffers (TLBs). Instructions in the instruction caches may be copies of instructions in memory 108 or storage 110, and the instruction caches may speed up retrieval of those instructions by processor 106. Data in the data caches may be copies of data in memory 108 or storage 110 for instructions executing at processor 106 to operate on; the results of previous instructions executed at processor 106 for access by subsequent instructions executing at processor 106 or for writing to memory 108 or storage 110; or other suitable data. The data caches may speed up reading or writing operations by processor 106. The TLBs may speed up virtual-address translation for processor 106. In particular embodiments, processor 106 may include one or more internal registers for data, instructions, or addresses. This disclosure contemplates processor 106 including any suitable number of internal registers, where appropriate. Where appropriate, processor 106 may include one or more arithmetic logic units (ALUs); be a multi-core processor; or include one or more processors 106. Although this disclosure describes and illustrates a particular processor, this disclosure contemplates any suitable processor or any combination of two or more processors.

In particular embodiments, memory 108 includes main memory for storing instructions for processor 106 to execute or data from subsystems 102 for processor 106 to operate on. As an example, and not by way of limitation, computer system 104 may load instructions from storage 110 or another source (such as, for example, another computer system 104) to memory 108. Processor 106 may then load the instructions from memory 108 to an internal register or internal cache. To execute the instructions, processor 106 may retrieve the instructions from the internal register or internal cache and decode them. During or after execution of the instructions, processor 106 may write one or more results (which may be intermediate or final results) to the internal register or internal cache. Processor 106 may then write one or more of those results to memory 108. In particular embodiments, processor 106 executes only instructions in one or more internal registers or internal caches or in memory 108 (as opposed to storage 110 or elsewhere) and operates only on data in one or more internal registers or internal caches or in memory 108 (as opposed to storage 110 or elsewhere). One or more memory buses (which may each include an address bus and a data bus) may couple processor 106 to memory 108. Bus 116 may include one or more memory buses, as described below. In particular embodiments, one or more memory management units (MMUs) reside between processor 106 and memory 108 and facilitate access to memory 108 requested by processor 106. In particular embodiments, memory 108 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 108 may include one or more memories 108, where appropriate. Although this disclosure describes and illustrates a particular memory, this disclosure contemplates any suitable memory.

In particular embodiments, storage 110 includes mass storage for data or instructions. As an example, and not by way of limitation, storage 110 may include a hard disk drive (HDD), a floppy disk drive, flash memory, an optical disc, a magneto-optical disc, magnetic tape, or a Universal Serial Bus (USB) drive or a combination of two or more of these. Storage 110 may include removable or non-removable (or fixed) media, where appropriate. Storage 110 may be internal or external to computer system 104, where appropriate. In particular embodiments, storage 110 is non-volatile, solid-state memory. In particular embodiments, storage 110 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 110 taking any suitable physical form. Storage 110 may include one or more storage control units facilitating communication between processor 106 and storage 110, where appropriate. Where appropriate, storages 110 may include one or more storages 110. Although this disclosure describes and illustrates particular storage, this disclosure contemplates any suitable storage.

In particular embodiments, I/O interface 112 includes hardware, software, or both, providing one or more interfaces for communication between computer system 104 and one or more I/O devices. Computer system 104 may include one or more of these I/O devices, where appropriate. One or more of these I/O devices may enable communication between a person and computer system 104. As an example, and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 112 or them. Where appropriate, I/O interface 112 may include one or more devices or software drivers enabling processor 106 to drive one or more of these I/O devices. I/O interface 112 may include one or more I/O interfaces 112, where appropriate. Although this disclosure describes and illustrates a particular I/O interface, this disclosure contemplates any suitable I/O interface.

In particular embodiments, communications interface 114 includes hardware, software, or both providing one or more interfaces for communication (such as, for example, packet-based communication) between computer system 104 and one or more other computer systems 104 or one or more networks. As an example, and not by way of limitation, communications interface 114 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communications interface 114 for it. As an example, and not by way of limitation, computer system 104 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 104 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 104 may include any suitable communications interface 114 for any of these networks, where appropriate. Communications interface 114 may include one or more communications interfaces 114, where appropriate. Although this disclosure describes and illustrates a particular communication interface, this disclosure contemplates any suitable communication interface.

In particular embodiments, bus 116 includes hardware, software, or both coupling components of computer system 104 to each other. As an example and not by way of limitation, bus 116 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 116 may include one or more buses 116, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Data obtained by the subsystem information consolidation engine 118 from the one or more subsystems 102 is consolidated and converted to usable data, which may comprise one or more machine learning algorithms. The consolidated information is processed by the processor 106, and sent to a viewing framework decision engine 122, which decides how to display the information in a tailored viewing framework, based on one or more parameters. The parameters could relate to manual preferences, entered by a user (such as which results or data they wish to see), or automatically detected parameters detected by the computer system 104 (such as security factors, hospital guidelines, the type of device that the viewing framework will be sent to, etc.). The viewing framework decision engine 122 sends the viewing framework to one or more display elements 120, preferably with little to no delay upon detecting the parameters.

Viewing parameters may comprise, but are not limited to, one or more of: a display device type, a user's credentials, and a location of the display device, manually entered user preferences, environmental conditions, and the like.

In an embodiment, the subsystem information consolidation engine 118 is capable of contextualizing a plurality of different information types, including numerical, verbal, measured, written, automatically received, and manually entered. The subsystem information consolidation engine 118 may comprise one or more machine learning and/or large language models, for sorting and contextualizing the information received from the subsystems 102 before sending it to the processor for processing. EHR systems allow institutions to set thresholds for lab results and vitals. Using these existing systems, HCPs typically receive notifications whenever a value exceeds a set threshold, often resulting in excessive alerts. The disclosed systems and methods comprise contextualizing the plurality of different information types involves taking into account individual patient baselines which may be impacted by comorbidities and risk factors, allowing for more accurate, relevant and personalized alerts and preventing notification fatigue. HCPs may set up custom thresholds for lab results and vitals their patients during onboarding or at later stages, which allows other members of the circle of care to know exactly when patient care should be escalated. The custom thresholds may comprise a critical low value, critical high value, and the corresponding unit for each lab result or vital element.

Patient onboarding is streamlined through automatic EHR integration that pre-populates data, minimizing manual input and reducing the risk of errors. To initiate the onboarding process, HCPs may input the medical record number (MRN) of a patient into the disclosed system to retrieve patient data via EHR integration. If patient data is found, HCPs are prompted to confirm the pre-populated information. Otherwise, HCPs may be prompted to re-enter the MRN or add a manual entry instead. In some embodiments, a plurality of patients may be onboarded at the same time, and an indicator may be present to show the status of the EHR data retrieval. For patients that have undergone surgery, HCPs may select or input an index surgery or reference surgery. The index surgery is required to select the correct input for the risk calculators. Once the index surgery is selected, additional operative details that could not have been pulled from EHR may be entered. The additional operative details may comprise surgery and procedure details (e.g., type of surgical procedure, start and end date and time, lead surgeon, blood transfusion status, ASA score), preoperative risk factors (e.g., demographics, medical history, surgical history, family history), intraoperative risk factors (e.g., organ texture and size, anesthetic management, hemodynamic factors) and postoperative status and disposition. In addition, custom patient thresholds for lab tests and vitals may be entered, allowing for clinical context to be considered. The patient's circle of care is also entered during onboarding from EHR or manual entry to determine each HCP involved in taking care of the patient (e.g., primary care physician, fellow or resident, ward physician, ICU physician, other specialists, etc.). Other inputs to the patient's circle of care may include an inpatient location and bed assignment.

HCPs may prioritize high risk patients through a triage feature on the displayed dashboard. HCPs may monitor the lab test results, vitals, inputs, outputs, as well as access operative details through the patient view, wherein abnormal values are clearly indicated through colour coding. HCPs may review different biological systems of the patient through the system view. In the system view, the risk of different complications according to each biological system is shown. In some embodiments, existing risk calculators may be used, and a selection of the existing risk calculators may be available to HCPs. HCPs may view the risk calculator inputs obtained via EHR or manually edit the entries if necessary. Additionally, HCPs may review the source of the risk calculators, risk factors, signs and symptoms, diagnostic reports, lab test results, and medications associated with each biological system.

In an embodiment, healthcare professionals 124, 126, and/or one or more hospitals 128, 130, may have access to one or more display elements 120 connected to a network 132. Information received, processed, and displayed may be shared amongst these groups over the network 132. The information may be anonymized prior to sharing it amongst the groups. The groups may be connected to one another based on similar flags in the information that they have sent to the computer system 104. Healthcare professionals 124 may be able to interact with the computer system 104 via a chat bot function, to request that it sends them more information, or connect them with another healthcare professional 126, relating to data that they enter into the system. Healthcare professionals 124, 126 may be able to interact with the computer system 104 to facilitate operational factors such as scheduling, treatment plans, ordering tests, adding medications, or developing a clinical study by entering information into the computer system 104, the operational factors being shareable with other members of the hospitals in which they are affiliated with 130, 128. The one or more hospitals 128, 130 may also alternatively represent other healthcare facilities such as clinics and specialized centers.

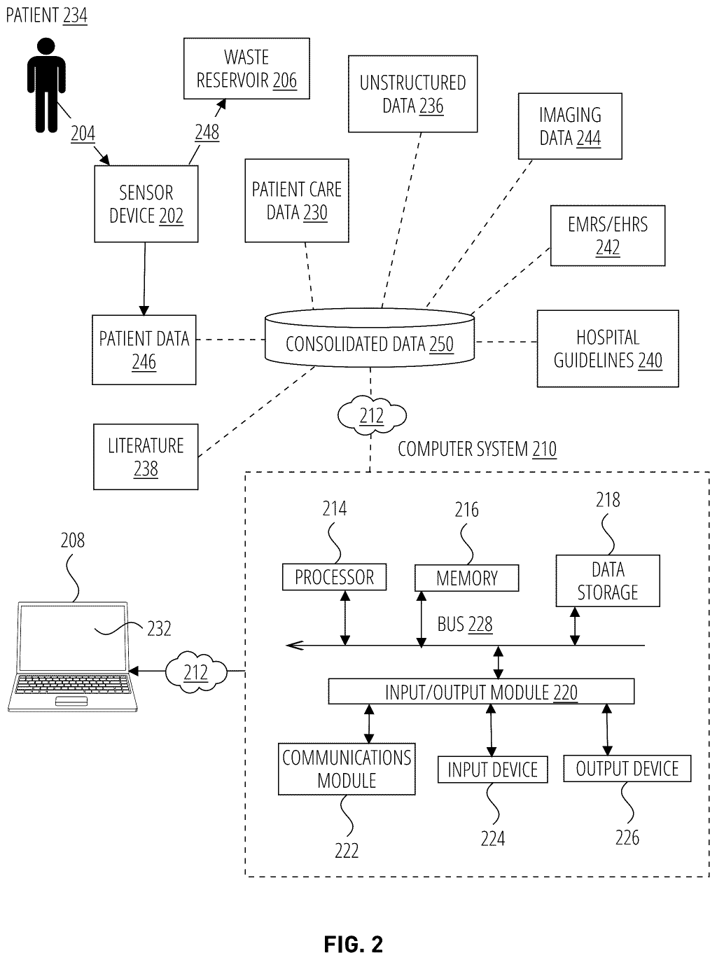

FIG. 2 is a block diagram illustrating a more detailed computer system 210 with which aspects of the disclosure may be implemented. Subsystems 102 shown here comprise one or more of: patient data 246 (measured from a patient 234 by one or more sensor devices 202), patient care data 230, unstructured data 236, EMRs/EHRs 242, hospital guidelines 240, literature 238, radiology data, imaging data 244, sensor data, surgical instrument data, and the like, may be inputted (manually or automatically extracted by the computer system), consolidated by the subsystem information consolidation engine 118 to generate consolidated data 250. The consolidated data 250 is sent to a processor 214 of the computer system 210 via a communication mechanism 212, where the consolidated data 250 is processed by a processor 214. The processed data is displayed on a display element 208 according to a pre-determined (by the viewing framework decision engine 122) viewing framework 232 for an HCP.

In an embodiment, fluid flows from a patient 234 (or from a fluid source comprising one or more standard samples, during calibration) to a sensor device 202 at step 204, where it flows over one or more sensor elements in the sensor device 202. Data pertaining to bioanalytes in the fluid is measured by the sensor elements and sent to the processor 214 of the computer system 210, where the patient data 246 is processed, or a calibration process is performed. Once the fluid is measured by the sensor device, the fluid flows to a waste reservoir 206 at step 248.

In an embodiment, non-fluid bio patient data pertaining to heart rate, blood pressure, respiratory rate, etc. may also be measured by one or more sensor devices 202 from a patient 234, such as a blood pressure cuff, a medical wearable, a smart watch, or video monitoring. Patient data 246 may be sent to the computer system 210 automatically or manually by an HCP through a communication mechanism 212. Alternatively, or in combination, patient care data 230 may be entered manually through an HCP scanning notes, typing in notes, filling out a form, extracting a patient's EHR, and the like.

Patient care data 230 may be entered by an HCP or automatically extracted by the computer system 210 (for example upon detecting that an HCP has added notes to a patient's clinical notes, extracting the notes, and sending the data from the notes to the processor 214 for processing the data).

A viewing parameter may be what type of display element 208 is being used to display information. If the viewing framework decision engine 122 detects that the display element 208 is a point of care display device, connected to at least one of the one or more medical devices, the viewing framework 232 may further comprise displaying one or more prompts to guide the user through a calibration of the at least one medical device. Point of care devices may be configured to display real-time data.

If the viewing framework decision engine 122 detects that the display element 208 is remote from a point of care, and the viewing framework 232 may further comprise further displaying, on the display device, patient condition data for each patient of a plurality of patients, and the processor 106 may convert measured data from each patient of a plurality of patients into a risk assessment for each patient. The risk assessment may be numerical (quantitative), or otherwise, qualitative assessments of risk may be more easily communicated to a healthcare professional. Remote devices may be configured to display trends in data.

Preferably, the viewing framework 232 further performs one or more of the following functions: updates and displays risk assessments related to the patient, sorts risk assessment based on level of risk, alerts certain device types (POC or mobile) that a patient is in need of immediate care. Alerts can be visual (a notification, for example), or auditory. A doctor may receive an automatic phone call playing an automated message, when a patient is at risk, for example. In some embodiments, the risk assessment may self-update using one or more risk calculators. The risk calculators may take into account a plurality of postoperative complications as shown in FIG. 8.

The viewing framework 232 may be interactive. An HCP may request an update (visual or auditory) from the viewing framework, whether or not there is an emergency. The viewing framework 232 may also display prompts for measuring specific vitals, prompts for taking tests at a given time, the prompts updated and displayed based on user's current, and anticipated/predicted condition(s) and a database with other users' condition(s) and risk values, coupled with an AI engine.

The viewing framework 232 may display different parameters based on the location of the patient. The viewing framework decision engine 122 may detect that a patient is in the ICU, for example, and update healthy/not healthy thresholds based on ICU guidelines (which may be different than those in the general ward, for example). The location of the patient may be departments within a given healthcare institution or across a plurality of different healthcare institutions. Preferably, the viewing framework 232 is adaptable to user inputs. Health care practitioners may interact with the system to view further details, input data etc.

Subsystems 102 may comprise: medical records, medical devices, electronic health records, among others. Data may be obtained at any one or more of the following points in a patient's stay: pre-surgical, intra-surgical, and post-operation, including post-discharge/remote monitoring data (for example, from monitoring devices continuously monitoring the patient and sending measured data over a network for processing).

Various data may be used throughout the patient journey to inform patient care: pre-operative data (such as age, weight, or medical history) may be processed to give a surgical plan, with additional, post operative plans including things like nutrition planning, medication, exercise, and the like, for optimal recovery, and also to inform intra- and post-op risk.

During the patient stay (intra-op), more data is continuously obtained from medical instruments, clinical notes, monitoring tools, and the like, which can be inputted for processing (in combination with pre-op data) to continuously update the post-op plan, as well as predicting post-op risk.

Post-operatively, patients may be monitored continuously or at regular intervals, at the point of care, for example, to continuously update risk predictions (in combination with already processed pre- and intra-op data), as well as potentially updating the patient care plan and/or suggesting interventions. Post-operatively, and post-discharge, the patient may be monitored remotely from the hospital, with a monitoring device, whose data may be processed continuously to update risk predictions and potentially alert the patient if they are at risk and must return to the hospital.

The viewing framework 232 may automatically communicate and display risk assessment (or trends in risk assessment) at different milestones (for example every hour post-surgery). Milestones may be preset by a user, or automatically determined by one or more machine learning algorithms, which determine the best times or instantaneous conditions at which a risk condition should be determined and communicated. The viewing framework 232 may also display: Periodic and automated generation of population health reports, catered/specific to a patient subgroup, healthcare professional, department, or similar. Preferably, the viewing framework 232 comprises an easy-to-access client portal for healthcare professionals.

Different visual techniques may be used to signal abnormalities and critical points in data, such as color coding the results (red for high risk, green for low risk, etc.). Alternatively, or perhaps in combination, the viewing framework may be configured to display a homunculus or a 2D or 3D image of the human body, illustrating (with colors, lights, flags, tags) targeted areas of the body (head, abdomen, etc.) where complications are suspected.

The computer system 210 may be implemented using hardware or a combination of software and hardware, either in a dedicated server, integrated into another entity, or distributed across multiple entities. The computer system 210, or aspects of it, may be integral to the sensor device 202, or separate from it. The sensor device 202 may communicate with the computer system 210 via communication mechanisms 212 wirelessly over a network, or via a wired communication mechanism.

Computer system 210 (e.g., server and/or client) includes a bus 228 or other communication mechanism for communicating information, and a processor 214 coupled with the bus 228 for processing information. By way of example, the computer system 210 may be implemented with one or more processors 214. Processors 214 may reside in the sensor device 202, in the computer system 210, or both. Data may be pre-processed in the sensor device 202, for example by correcting data based on on-site conditions and then sent to a computer system 210 for more rigorous processing. Processor 214 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 210 can include, in addition to hardware, code that creates an execution environment for the computer program in question, e.g., code that constitutes processor firmware, a protocol stack, a database management system, an operating system, or a combination of one or more of them stored in an included memory 216, such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 228 for storing information and instructions to be executed by the processor 214. The processor 214 and the memory 216 can be supplemented by, or incorporated in, special purpose logic circuitry. The sensor device 202 may have its own memory 216 for storing data. The memory 216 may be in a separate computer system 210. The memory 216 may comprise cloud data storage 218.

The instructions may be stored in the memory 216 and implemented in one or more computer program products, i.e., one or more modules of computer program instructions encoded on a computer readable medium for execution by, or to control the operation of, the computer system 210, and according to any method well-known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 216 may also be used for storing temporary variable or other intermediate information during execution of instructions to be executed by processor 214.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 210 may further include data storage 218 such as a magnetic disk or optical disk, coupled to bus 228 for storing information and instructions. Computer system 210 may be coupled via input/output module 220 to various devices, including a display element 208, such as a phone screen or computer screen. The input/output module 220 can be any input/output module. Exemplary input/output modules 220 include data ports such USB ports. The input/output module 220 is configured to connect to a communications module 222. Exemplary communications modules 222 include networking interface cards, such as Ethernet cards and modems. In certain aspects, the input/output module 220 is configured to connect to a plurality of devices, such as an input device 224 and/or an output device 226. Exemplary input devices 224 include a keyboard and a pointing device, e.g., a mouse or a trackball, by which a user can provide input to the computer system 210. Other kinds of input devices 224 can be used for interaction with a user as well, such as a tactile input device, visual input device, audio input device, or brain-computer interface device. For example, feedback provided to the user can be any form of sensory feedback, e.g., visual feedback, auditory feedback, or tactile feedback and input from the user can be received in any form, including acoustic, speech, tactile, or brain wave input. Exemplary output devices 226 include display devices such as an LCD (liquid crystal display) monitor, for displaying information to the user.

According to one aspect of the present disclosure, the methods for processing analyte data and/or calibrating a system to process analyte data, detected by a sensor device 202, described herein, can be implemented using a computer system 210 in response to processor 214 executing one or more sequences of one or more instructions contained in memory 216. Such instructions may be read into memory 216 from another machine-readable medium or non-transitory computer readable medium, such as a data storage device 218. Execution of the sequences of instructions contained in the main memory 216 causes processor 214 to perform the process steps described herein. One or more processors in a multi-processing arrangement may also be employed to execute the sequences of instructions contained in memory 216. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., such as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 210 can include clients and servers. A client and server are generally remote from each other and typically interact through a communication network. The relationship of client and server arises by virtue of computer programs running on the respective computers and having a client-server relationship with each other. Computer system 210 can be, for example and without limitation, a desktop computer, laptop computer, or tablet computer. Computer system 210 can also be embedded in another device, including the sensor device 202, for example.

The term “machine-readable storage medium” or “computer readable medium” as used herein refers to any medium or media that participates in providing instructions to processor 214 for execution. Such a medium may take many forms, including, but not limited to, non-volatile media, volatile media, and transmission media. Non-volatile media include, for example, optical or magnetic disks, such as data storage device 218. Volatile media include dynamic memory, such as memory 216. Transmission media include coaxial cables, copper wire, and fiber optics, including the wires that comprise bus 228. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

As the processor 214 reads and processes patient data (whether from monitoring patient fluid, calibration fluid, cleaning fluid, manually input or a combination thereof) or patient care data 230, or similar, information may be read from the data and stored in a memory device, such as the memory 216. Additionally, data from the memory 216 may be accessed at a customer server, via a network, such as the bus 228, in order to view bioanalyte concentrations and/or any risk assessments, or changes in patient care or scheduling, determined by the processor 214. Further, data storage 218 may be read and loaded into the memory 216. Although data is described as being found in the memory 216, it will be understood that data does not have to be stored in the memory 216 and may be stored in other memory accessible to the processor 214 or distributed among several media, such as the data storage 218, and may be received at a remote or local server for data processing.

Machine learning algorithms may be applied to data acquired by any one of the systems and subsystems disclosed herein. For example, AI may be employed to extract relevant information from free-text clinical notes to summarize the patient's condition on the bedside display's viewing framework 302. Further, the memory 108 or 216 stores a plurality of data obtained from HCPs and hospitals globally, enabling collaboration between researchers both within the hospital and across institutions globally.

Consolidation of data via the subsystem information consolidation engine 118 ensures that previously unusable (particularly quantitatively), and therefore unusable data, such as clinical notes, literature, billing information, and the like, combined with EHRs and other electronic information extracted from a hospital, means that AI engines are able to make more accurate and contextually relevant conclusions and decisions.

These machine learning algorithms may include, for example, deep learning architectures such as Deep Belief Network (DBN), Stacked Auto Encoder (SAE), Convolutional Neural Network (CNN) or Recurrent Neural Network (RNN) may be used. Other examples include, without limitation, Restricted Boltzmann machines (RBM), Social Restricted Boltzmann Machines (SRBM), Fuzzy Restricted Boltzmann Machines (FRBM), TTRBM models of Deep Belief Networks (DBN) or similar approaches could be used; AE, FAE, GAE, DAE, BAE models of Statistically Adjusted End Use (SAE) models could be used; models such as AlexNet, ResNet, Inception, VGG16, ECNN models of CNN may be used; Bidirectional Recurrent Neural Networks (BiRNN), Long Short-Term Memory (LSTM) networks, Gate Recurrent Unit (GRU) of RNN may also be used. Additional techniques specific to time-series modelling may be employed including, but not limited to, dynamic time warping, change point detection, and Autoregressive Integrated Moving Average (ARIMA).

In some embodiments, other types of algorithms such as physics-based mathematical computations and basic multiple linear regression models may also be relied upon in conjunction with or in complementarity with those architectures and learning algorithms. This may further include cumulative average (CA) methods.

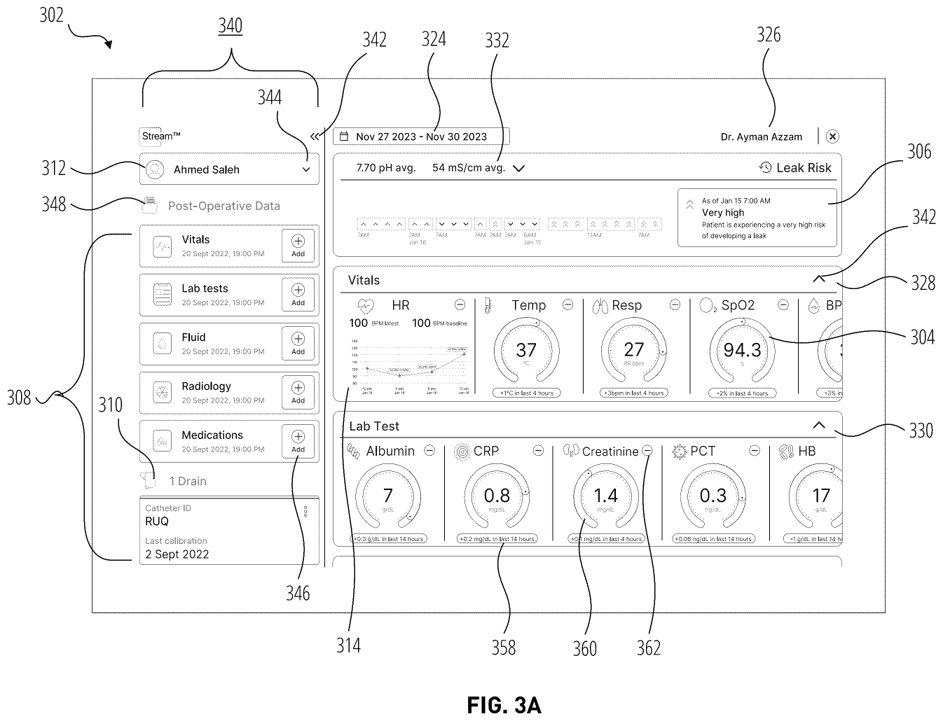

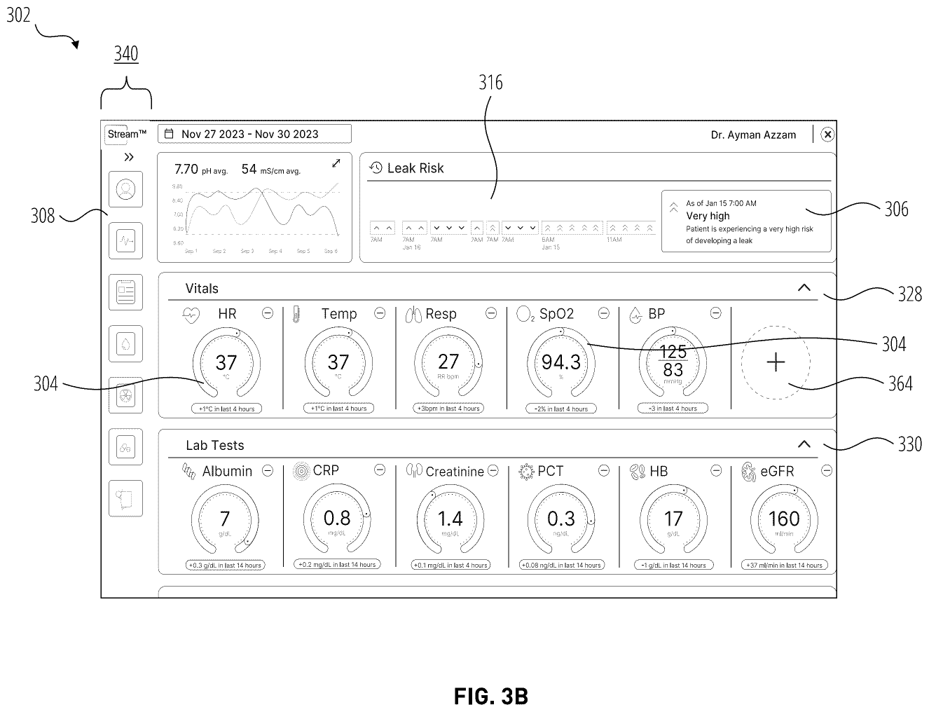

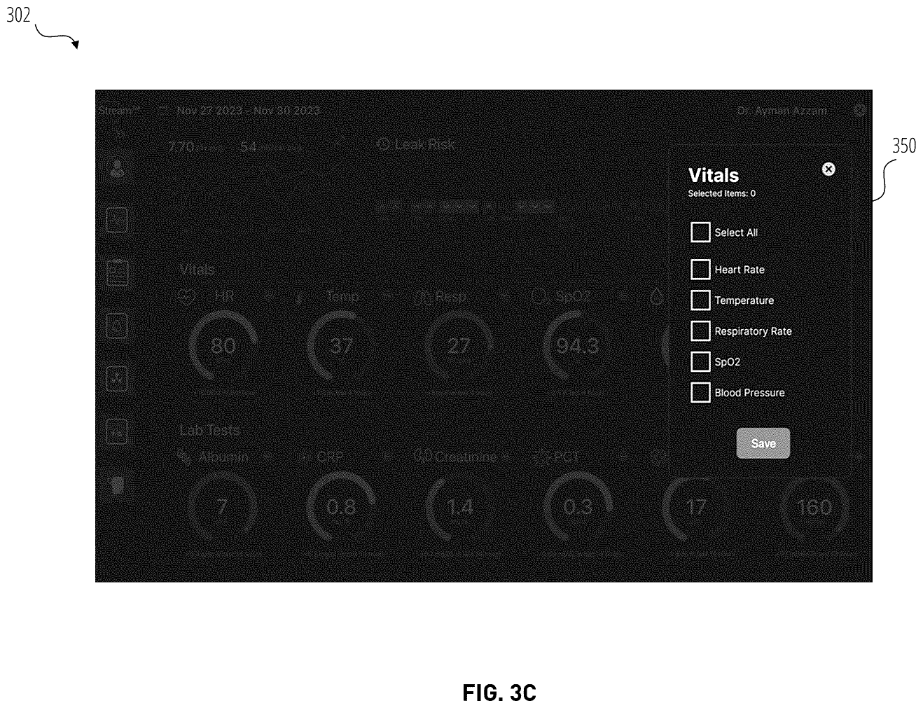

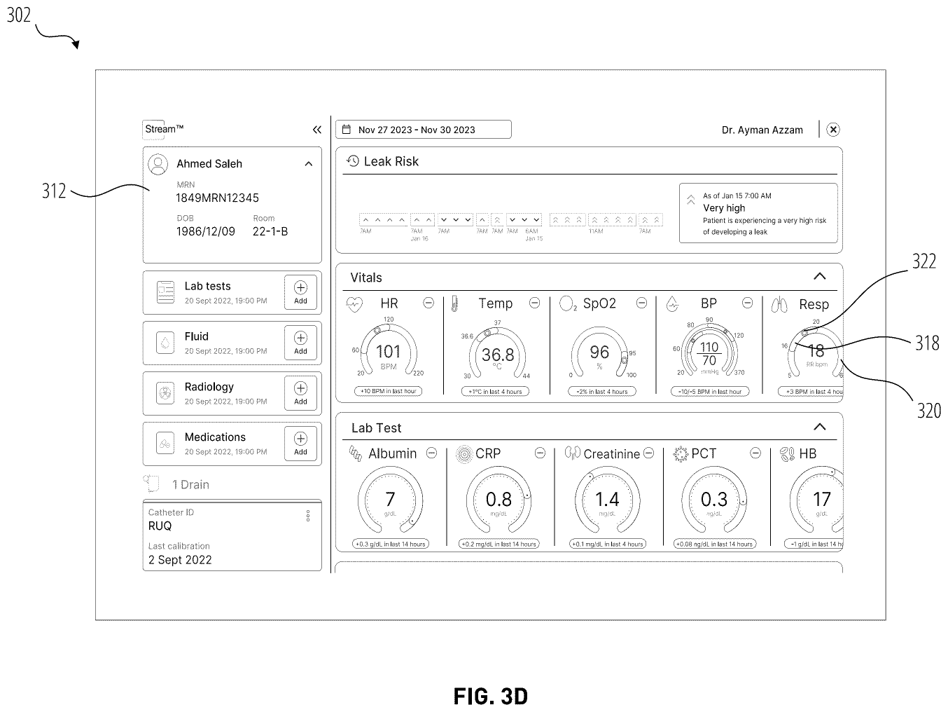

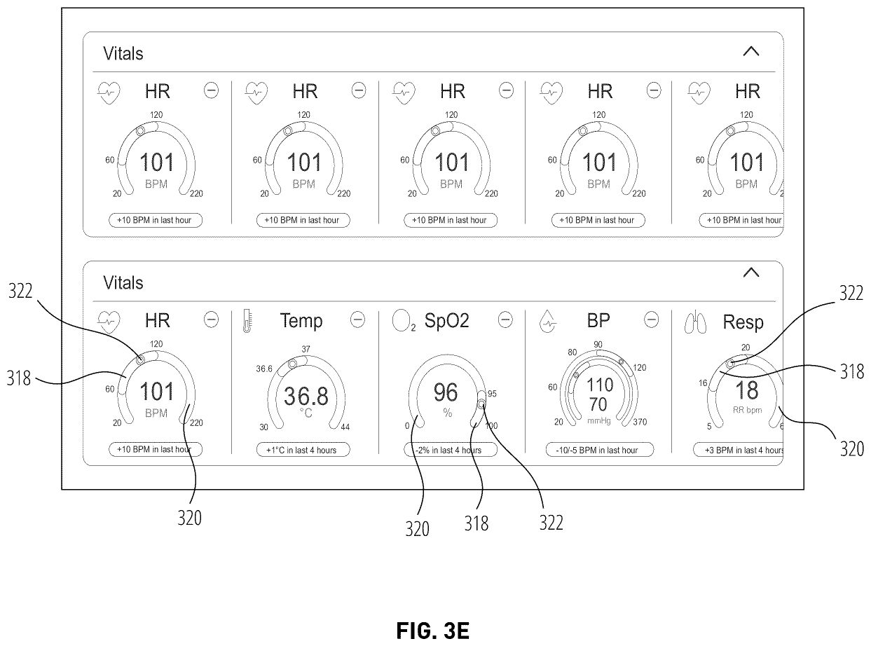

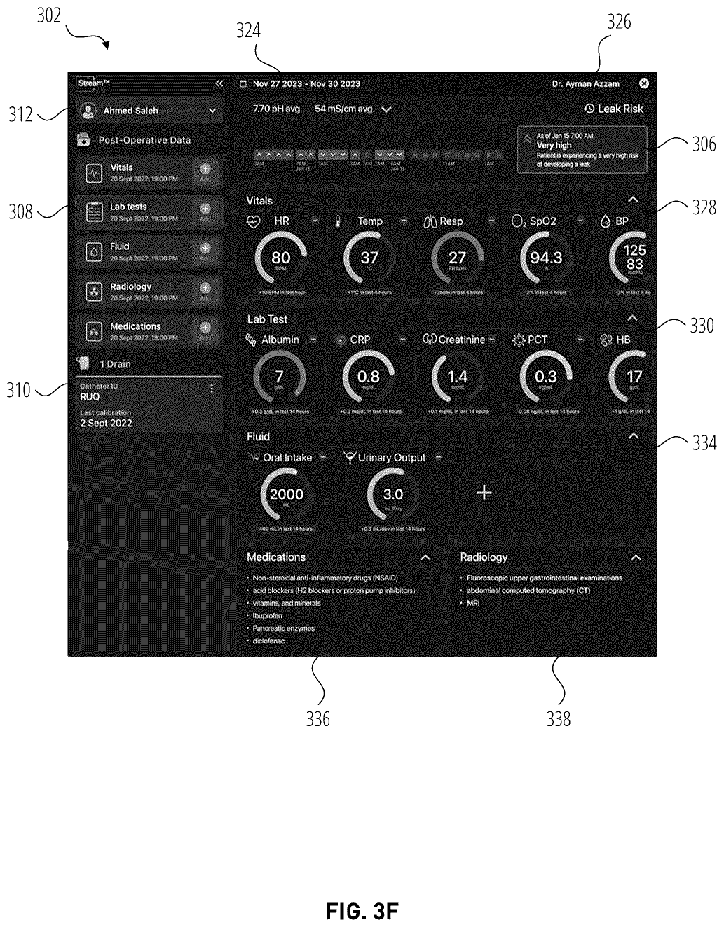

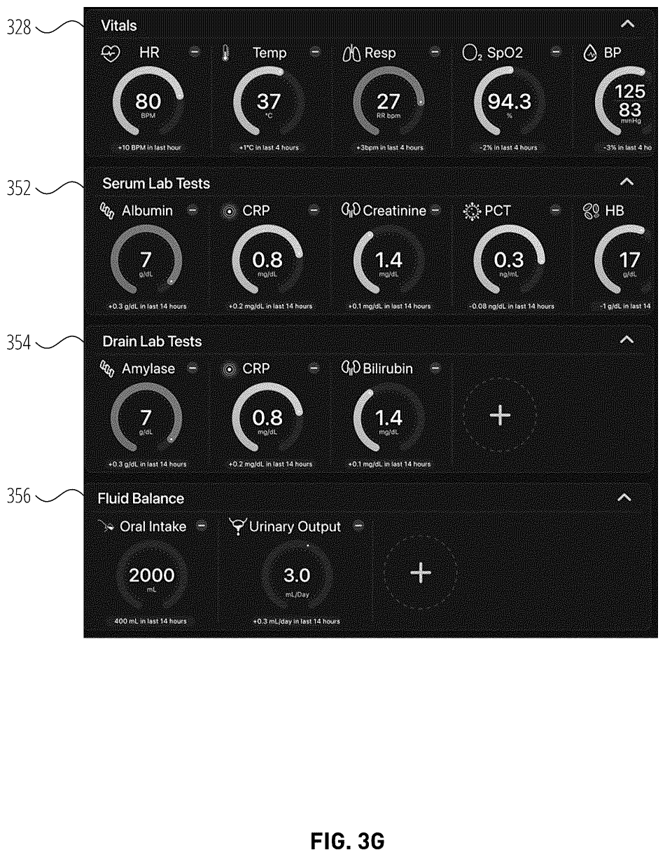

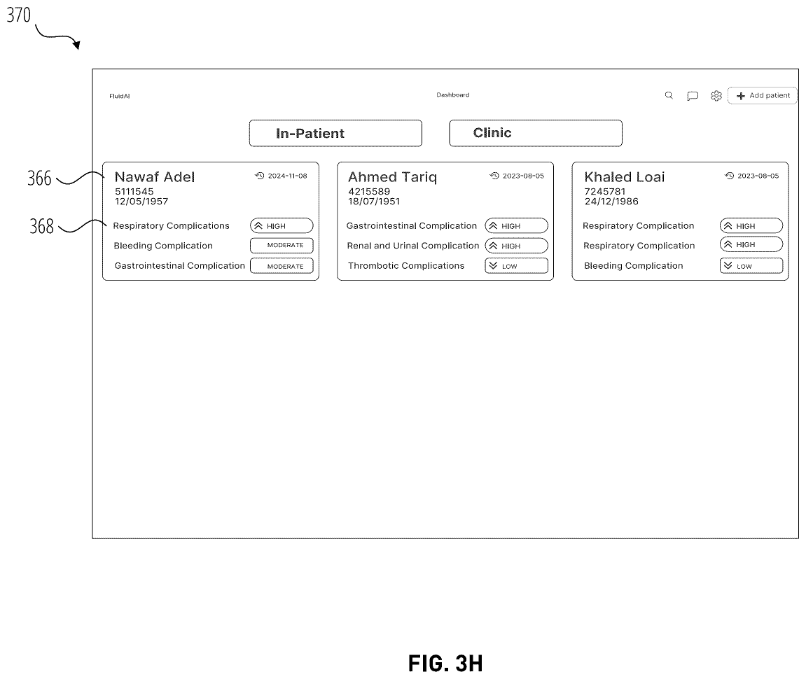

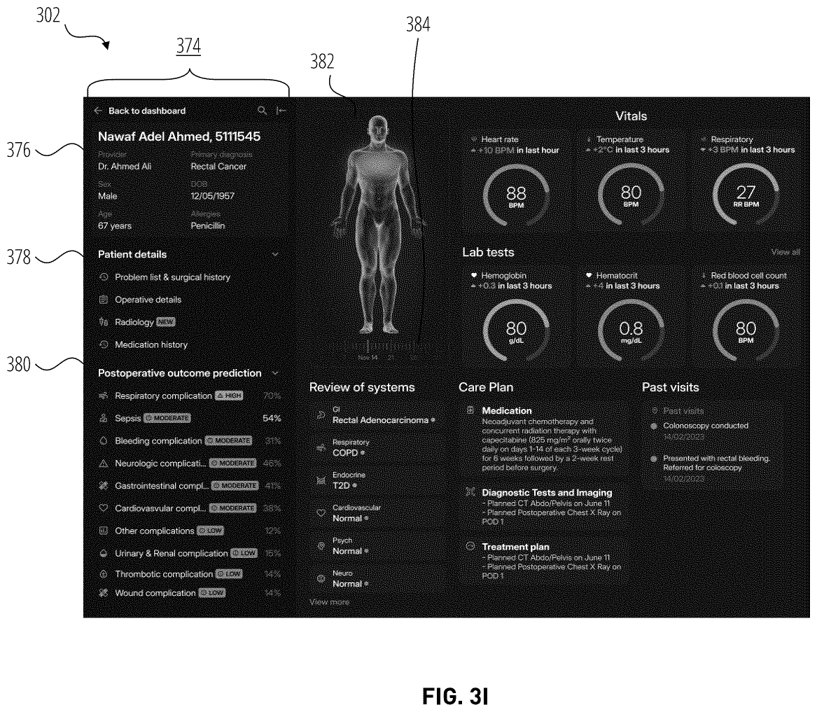

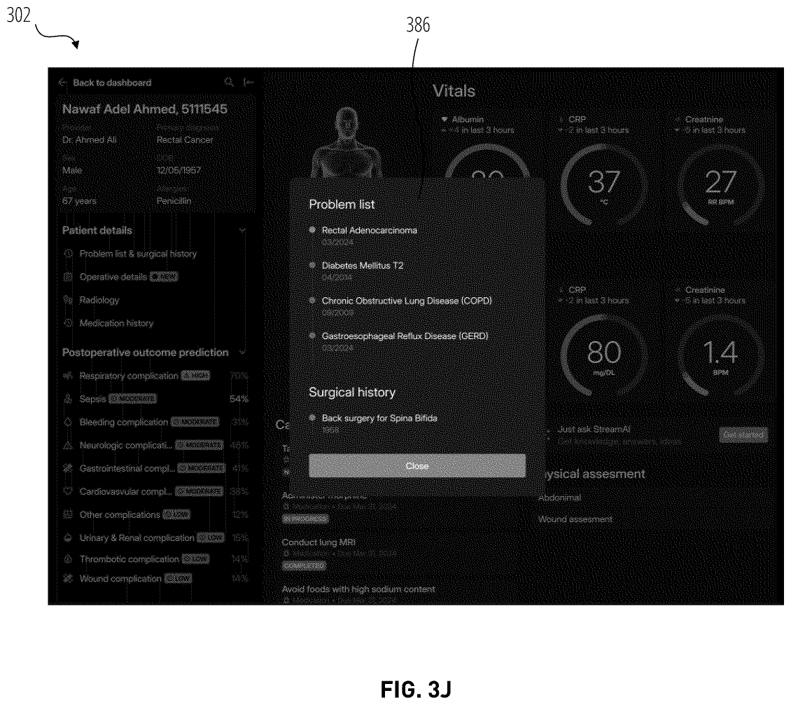

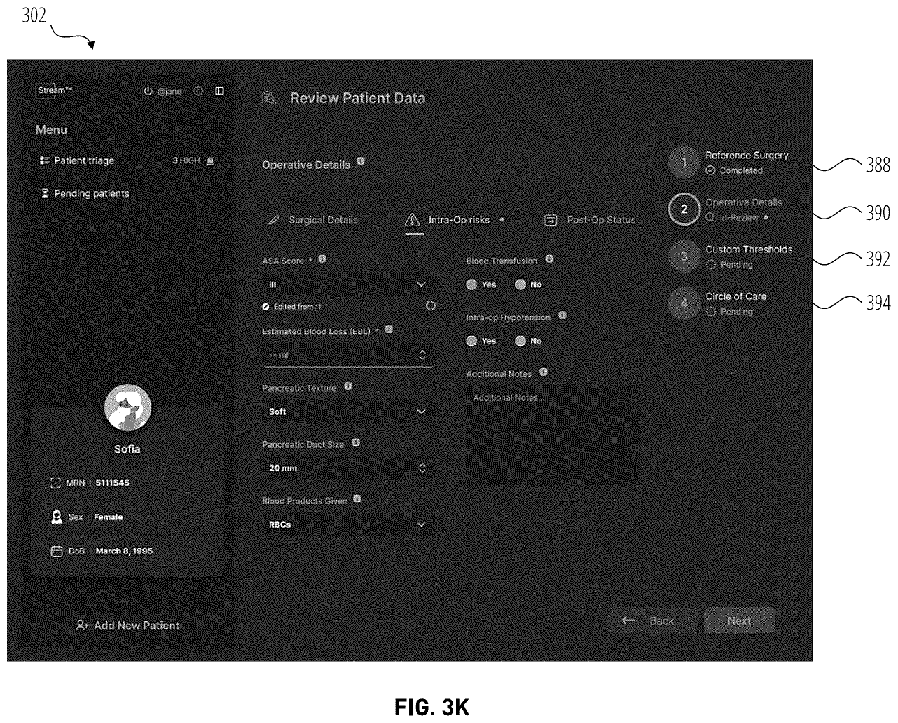

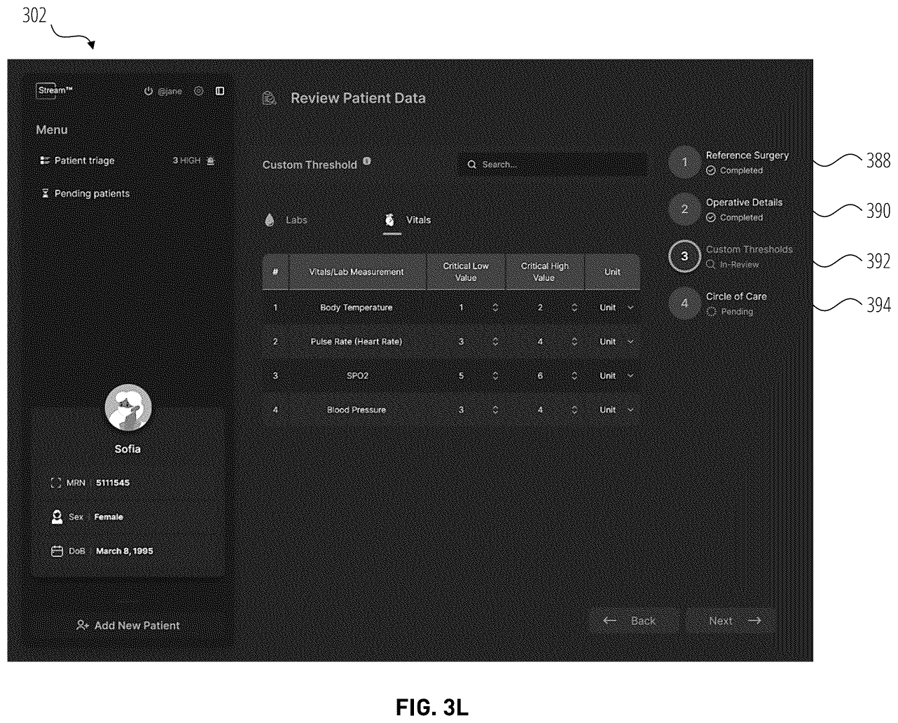

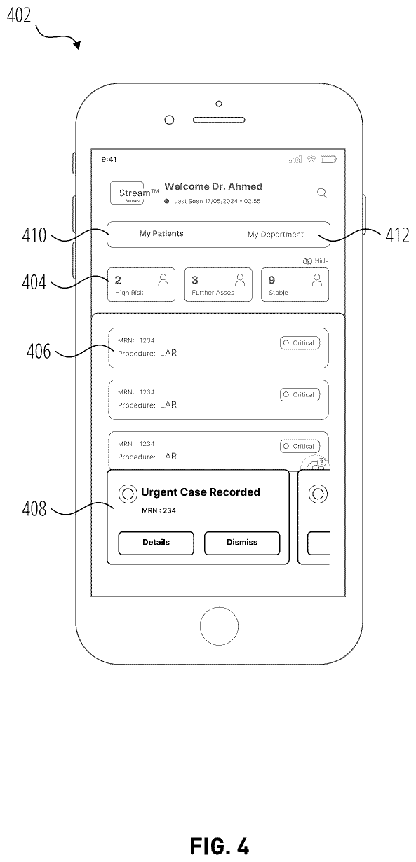

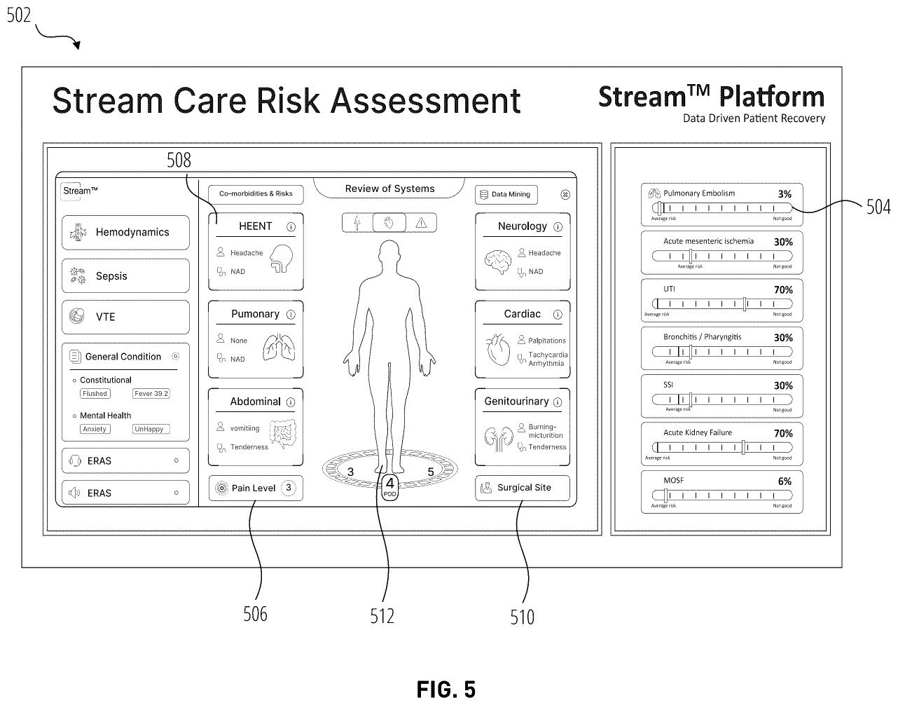

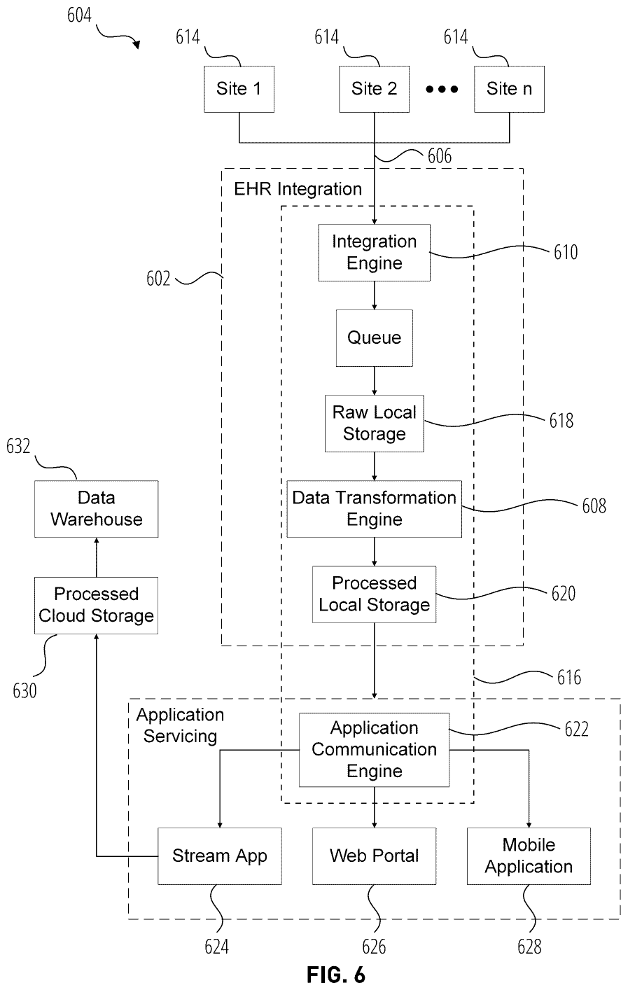

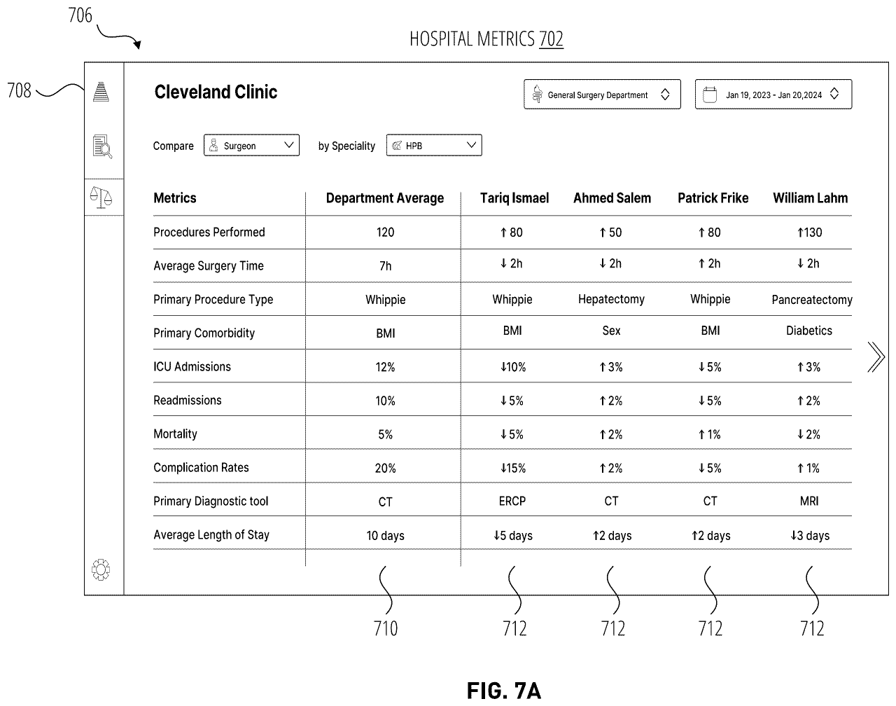

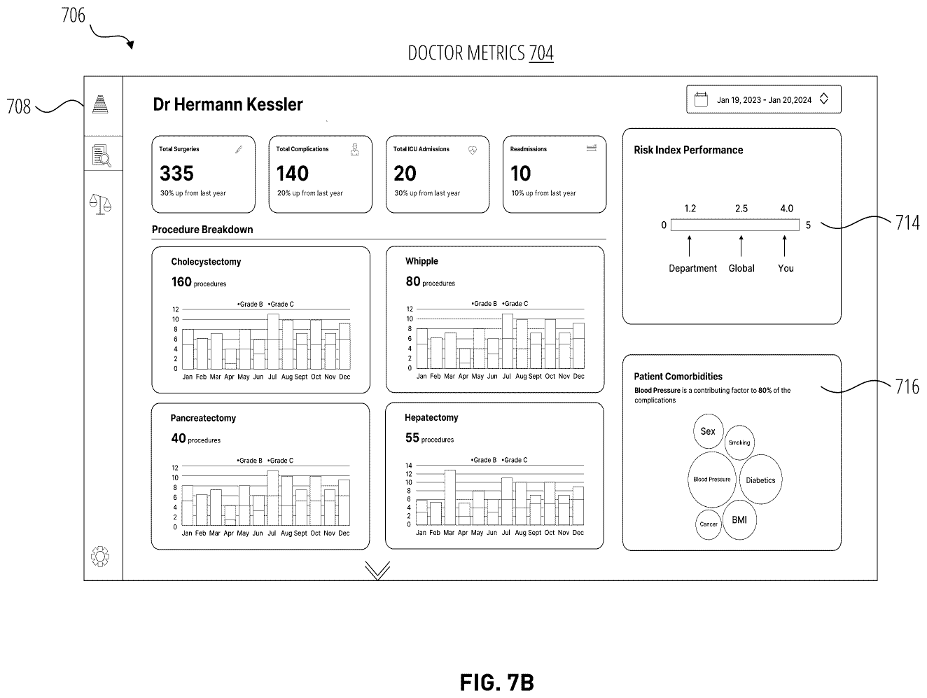

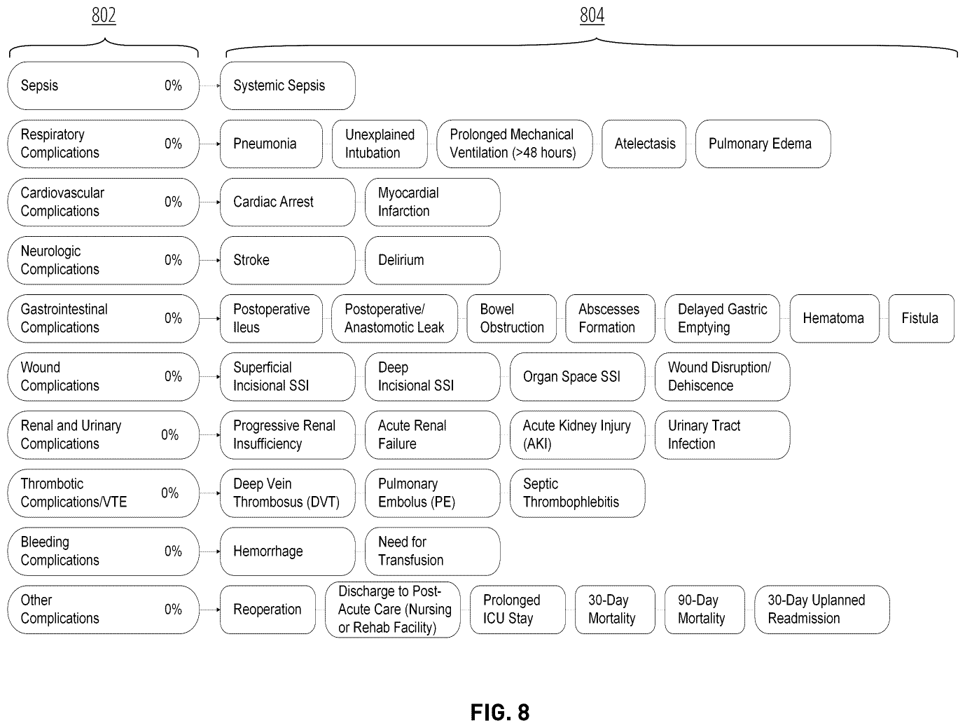

FIG. 3A illustrates an exemplary viewing framework 302 provided by the systems and methods of the present disclosure. Systems and methods disclosed herein solve multiple technical problems faced by healthcare practitioners in modern hospital settings. Systems and methods disclosed herein expedite, digitize, and streamline patient care by synthesizing all sources of data into measurable, and then interpretable information.

Systems and methods disclosed herein are well suited for use in post-operative care settings for patients that have undergone surgery where anastomotic leak risks, or other post-surgical risks, are of concern. The systems and methods as disclosed herein may be readily adapted for any one or more of patient monitoring at the following points: pre-operative, intra-operative, and post-operative (for example post discharge of the patient). The patient may be monitored before, during, and after, their stay in a hospital, for increased predictive accuracy of the models used to make risk assessments.

The system is designed to be user-friendly, allowing for the ease of viewing and interpretation through the latest readings and trends relating to one or more of: vital signs, lab tests, fluid readings, medications, radiology tests and imaging tests. One or more of the above may be displayed on a display viewing framework 302 that is interactable through: adding or removing relevant vital signs, lab tests and fluid readings, expanding views to see graphical trends, customizing ‘block items’ to only display relevant or important data.

The system may be used by HCPs in a pre, intra, or post-operative (and/or post-discharge) care setting. HCPs may be one or more of, but are not limited to, OR/OT, Nurse, Attending Surgeon, Resident Surgeon, On-call Physician (ICU or Ward). HCPs may use the system a post-operative setting (most likely in the ICU) to input the relevant data fields that require manual entry, while some tests and/or vitals may be extracted directly from a sensor device or other subsystem (such as existing EHR systems).

The system may be supported on one or more operating systems, including but not limited to: Android™, iOS®, Unix®, ChromeOS™, macOS®, Linux®, Windows®, and the like. The system may comprise, in the background, several other components and other software needed to run it on a device (such as a mobile phone, a desktop or laptop computer, or a tablet). The system may comprise, and/or interact with: (i) a Cloud system with appropriate database schemas to store the saved data fields; (ii) application programming interfaces (API) to send patient data to the Cloud; and (iii) a display element which displays the viewing framework 302.

It is important to note that all features of the system may be built as a stand-alone module application in a separate repository and can be invoked as an independent app within a separate software module.

The viewing framework 302, displayed on a user interface, uses a set of visual user interfaces that have been designed and are presented in this section in addition to requirements describing the required functionality. The viewing framework as shown in FIG. 3A to FIG. 3D illustrates different aspects of a cockpit view for a single patient in accordance with one embodiment. The viewing framework 302 further comprises a collapsible panel 340 having patient & device details. If the user taps on the patient demographics button 344, the patient details showing the demographics of the patient shall be shown. For example, patient data is shown in a drop-down menu to be displayed on the viewing framework 302 as in FIG. 3D. If the User taps on the add data button 346 from the patient data 348 options under the collapsible panel 340 having patient & device details, a data entry form field for the item shall pop up. The data entry form may be imported from an existing system. Data shall be saved in the appropriate database as indicated by the existing system.

The viewing framework 302 displays one or more patient specific readings and/or assessments, according to the parameters set by the user. Assessments may comprise the risk assessment 306, which may be displayed as trends in device readings, and an overall risk assessment, as shown in FIG. 3A. Vital signs 328 may comprise patient data relating to one or more of: heart rate, temperature, respiratory system, SpO₂, blood pressure, and the like.

Lab tests 330 may comprise one or more of: serum lab tests, drain lab tests, and the like (shown in more detail in FIG. 3G). Serum lab tests may show results relating to one or more of: albumin, c-reactive protein (CRP), creatinine, procalcitonin (PCT), hemoglobin (HB), estimated glomerular filtration rate (eGFR), and the like, while drain lab tests may show results relating to one or more of: amylase, CRP, bilirubin, and the like.

Each vital sign, lab test, and fluid reading may have an upper and lower healthy range set for the patient. The upper and lower bounds may be set automatically, based on baseline readings from the patient, or from an average of patients with similar demographics, or may be set manually by the HCP. The upper and lower bounds may also be set based on literature or hospital guidelines.

The viewing framework 302 displays, for each of the patient specific readings: (i) Display the latest reading taken in a gauges 304; (ii) Show a magnitude change 358 (+ or −) of the latest reading from the previous reading; and (iii) Assign a portion of the gauge 360 with a color based on whether the latest reading is within range of the upper and lower healthy bound. For example, display green if within the range, display red if significantly above or below the upper and lower bound, display amber if slightly above or below the upper and lower bound.

The viewing framework 232 may further display calibration instructions, upon receiving information relating to the calibration status of one or more connected devices 310 such as a drain. The calibration instructions may be displayed as an animation of the calibration steps. The animation may be colored. A progress bar indicating how far into the calibration process the user is, may be displayed. A loading level shall depend on the magnitude of the measured value relative to the absolute range.

The user may collapse results by tapping on the collapse button 342 on any one of the panes, which will minimize the pane to just display the pane's title with an expand button. If the user taps on anyone of the remove buttons 362 on the top right of a display square, the viewing framework 302 shall remove the block item e.g. for HR, the viewing framework 302 shall remove HR from the Pane View. If the user swipes or scrolls to the left, further block items may be displayed. Swiping may be bi-directional to view further block items. If the user taps on one of the block items with a patient measurement, a graph showing trends 314 (for example, the last three days of measurements), shall appear as shown for heart rate. The graph may show the upper and lower bound ranges in addition to the latest and previous data points.

For device readings 332, the viewing framework may display a graph of all of the device readings from all devices (or all sources). For example, a graph of all of the pH values, or all the conductivity values, measured by one or more connected devices 310, at all drains connected to the patient's surgical sites. The viewing framework 302 may further illustrate the latest device reading, with no classification if the value is good or bad. The displayed risk assessment 306 may illustrate a combination of factors measured or indicated by the connected devices 310, and vitals, lab tests, fluids, medications, or tests, which indicate the patient's risk of developing a complication. The medication list 336 and test list 338 as shown in FIG. 3F may require an integration with a hospital's Electronic Medical Records (EMR) system.

EMR systems may be digital, paper based, or some combination thereof (such as a scan), so the system may comprise further components to convert EMRs to usable information. Such components may comprise, but are not limited to, Optical Character Recognition (OCR), Natural Language Processing (NLP), and a combination thereof. The list of medications the patient is on may be displayed when extracted by the system from the EMR. The list of tests (for example, radiology scans) that has been ordered for the patient shall be displayed when extracted by the system. Preferably, the system shall be able to display and move to the next sections with no or little delay.

Protection of Sensitive Information (PHI) data is stored in accordance with HIPAA standards (and other similar privacy standards) and patients are de-identified in the Cloud. The system may further comprise an authentication or encryption workflow, password protection, or an encryption step, in order to further protect patient or healthcare professional data. Some events are tracked by the system using Firebase and stored in the Cloud service accordingly.

The system is designed for ease of use, and interpretation of results, requiring little to no training. The system may function with or without audible features and therefore its use shall not be affected by any hearing impairment of the user. The system uses some visual feedback based on colors. This feature is, however, not essential, as the colours represent data that is also displayed with text.

An HCP may also enter the surgery start and end time for display on the viewing framework 302.

Database Requirements: The system will send data to appropriately defined schemas in the database to store all input information on the patient during a patient onboarding workflow.

Installation and Operation: The system may be installed on a device, such as a computer, tablet, smartphone, and the like, as an app. For example, when installed on an Android system, the installation of the app is generally handled by the internal functionality of the Android Operating System. The deployment is enabled using a mobile device management (MDM) service, such as Esper™.

User Maintenance: Any updates to the system and its app are automatically done using the internal functionality of the MDM system. When a new version of the app is published, it is automatically downloaded and installed onto the users' device. When updates to the app are pushed, these will automatically reflect in the app.

Systems and methods disclosed herein may provide users with user-friendly and easily interpretable graphical representations of trends in vital signs, lab test results, and fluid readings, for example in the form of one or more gauges 304. The choice of display of vitals, lab tests, etc. may be configurable, for example, based on hospital guidelines.

In the illustrated example, the gauges 304 if the viewing framework 302 display real-time measurements of a patient's vitals, which may include, but are not limited to, one or more of: temperature, respiratory rate, oxygen saturation (SPO₂), blood pressure. In the illustrated example, the gauges 304 if the viewing framework 302 display real-time measurements of a patient's lab results, which may include, but are not limited to, one or more of: albumin, c-reactive protein (CRP), creatinine, procalcitonin (PCT), and hemoglobin (HB).

A feature of the viewing framework 302 is that the gauges 304 may update continuously, and in real time, if a patient is connected to a sensor device with one or more sensors, or patient monitoring device, communicating patient data to the system. The gauges 304 may update upon manual data entry, after an HCP has taken measurements of the patient's vitals, for example.

Within the viewing framework 302, a user, such as an HCP, may easily view trends 314 in a patient's condition by clicking on the gauges 304. The gauges 304 will flip to display a graph, illustrating the change in the patient data over time. The illustrated example shows the patient's heart rate trends over four measurements.

Within the viewing framework 302, a risk assessment 306 may be displayed. The risk assessment 306 comprises an assessment of whether the patient is at risk for developing a certain condition or experiencing a complication, or other metrics including, but not limited to: survivability, 30-day readmission, ICU readmission, risk for escalation of care, and the like.

The viewing framework 302 may also display the latest readings and allows users to add or remove items relevant to them in the display selection 308. The viewing framework 302 may also display one or more connected devices 310, such as sensor devices, or patient monitoring devices, for example. The viewing framework 302 also displays users with a list of medications and radiology tests (among others) the patient has in their treatment plan. The viewing framework 302 may further comprise a name for the patient's lead healthcare provider 326. The viewing framework 302 may also display a date selection range 324, for selecting the date for which the HCP would like to view data, and device readings 332, showing the latest readings taken by connected devices 310.

The viewing framework further comprises a Patient ID 312, which further comprises information relating to the patient whose data is displayed. The patient may be selectable from a list of patients being tended to by one or more HCPs. Healthcare providers may update the patient data, vitals, lab tests, medications, etc., that are specific to the patient ID, and the system may then update the viewing framework 302 accordingly.

The viewing framework may be customizable to each HCP ID and may update automatically upon the HCP entering log in credentials (such as a password, fingerprint, or an ID fob, or the like), so that the HCP does not need to customize their viewing framework 302 each time they go to work, for example.

These are all displayed in the main viewing framework 302, or ‘cockpit’ view of the system's dashboard providing HCPs with an all-encompassing view on the patient's status to make informed clinical decisions. Systems and methods as disclosed may solve the technical challenge of integrating with a plurality of hospital information management systems (digital or otherwise), and that of determining what is contextually relevant to an HCP for a particular patient at a particular moment of time. Prior art systems and methods were typically not adapted to a plurality of hospital systems, and the data provided was pre-set, and not based on metrics determining whether it was useful or otherwise.

The display framework operates separately and independently from other hospital data systems, in order to ensure compliance with regulatory requirements. For example, the viewing framework 302 shall be invoked as an independent application from other applications that are either Software as a Medical Device or non-Software as a Medical Device, without affecting the user experience.

This means that the two separate applications shall run and transition between each other in the background seamlessly without the user having to manually transition between one or the other.

FIG. 3B illustrates additional features of the viewing framework 302. The collapsible panel 340 with the display selection 308 in this embodiment is collapsed, such that the HCP may view an additional piece of processed data. Risk logic 316, which may comprise data that led to the system displaying a certain risk assessment 306, may be shown. This may be shown in terms of trends in the patient's data, and/or trends in the patient compared with one or more other patients of a sample population, under the same patient care conditions.

In the illustrated example, the viewing framework 302 illustrates a risk assessment 306 of “very high”, and to its left, the patient's pH trends are showing as rapidly decreasing, which may be indicative of a patient developing an anastomotic leak. Of course, it should be readily understood by one skilled in the art that the viewing framework 302 may also display a high, low, or medium risk value, and trends relating to that risk, of any condition determinable by the system's logic and database.

Such conditions and/or complications may include, but are not limited to, sepsis, anemia, infection, hemorrhaging, embolism, thrombosis, lung complications, urinary complications, anastomotic leaks, and chest tube leaks. If the user taps on the large add button 364 icon, a pop-up menu 350 shown in FIG. 3C shall be displayed allowing them to add in block items that are not yet displayed on the pane. In addition, the one of more gauges 304 may be interactive wherein the user may tap or click on the one or more gauges 304 which triggers the corresponding block item to switch views from the gauge view to a graph showing trends 314 in vital signs and/or lab test results, or vice versa. An example showing the capability of the viewing framework to switch between the gauge view and the graph view showing trends can be found within the heart rate block item under the vitals pane in FIG. 3A and FIG. 3B. In some embodiments, the one or more interactive gauges may specifically have a one-click function to flip the one or more interactive gauges to a trendline showing a trend in values.

FIG. 3D illustrates additional features of the viewing framework 302. In the illustrated embodiment, Patient ID 312 is expanded to view patient data such as their medical record number (MRN), date of birth (DOB), and room number. Other information may be displayed, including, but not limited to: weight, height, important notes, reason for visit, and the like. Patient data, such as MRN and DOB may be encrypted in the Cloud for additional security.

In the illustrated embodiments, the gauges 304 show healthy ranges 318 and unhealthy ranges 320 for vitals, and display where in those ranges the patient value 322 lies, along with the absolute value of the patient measurement. These features are further emphasized in FIG. 3E.

FIG. 3E is an exemplary view of the one or more gauges 304 having patient measurements that may be displayed by the viewing framework. Each gauge may also display a note on how the patient measurements have changed since the last measurement (or other useful trends). In some embodiments, gauges 304 may have colors corresponding to risk levels, or whether the measurements are within a desired range. For example, green may indicate that a patient's measurements are well within a desired range, yellow may indicate that a patient's measurements indicate they are starting to decline, and red may indicate that a patient's levels are well outside of the desirable range, and/or the patient is at risk due to their measurements.

FIG. 3F illustrates an exemplary embodiment of the viewing framework 302, wherein the viewing framework 302 comprises one or more of:
1. Patient name and details (Patient ID 312).
2. Interactive Post-Operative Data for Vitals, Lab Tests, Fluid, Radiology and Medications that the user can use to add the latest data points (shown as display selection 308).
3. Connected devices 310, such as drains, catheters, and the like, for devices connected to the patient, which may be measuring patient data, or which may provide information about the patient's post-surgical needs.
4. A Date selection range 324 which may be customizable, and the range may date back to the patient's entire history with the hospital, or as short as one day. The date selection range may have a default range, i.e. today's date back 3 days. Changing the date selection shall automatically change the data displayed according to the selected range.
5. A lead HCP 326. The lead HCP may be, for example, the lead surgeon or the patient's primary nurse. This may further comprise the HCP's contact information.
6. Latest device readings 332 of patient data, such as conductivity and pH readings, which may be measured by one or more connected devices 310.
7. Risk assessment 306, determined based on the one or more readings, by a processor of the system.
8. Vital signs 328, showing the latest vitals readings for each.
9. Lab tests 330, showing the latest readings for each.
10. Fluid readings 334, showing the latest readings for each.
11. A medication list 336, listing the medications the patient is taking, and (optionally) the schedule for taking the medications.
12. A test list 338, listing the tests the patient is undergoing (for example, but not limited to, radiology tests).

FIG. 3G illustrates additional panels and patient measurements that may be added to the viewing framework 302. Patient measurements may comprise: vital signs 328, serum lab tests 352, drain lab tests 354, and fluid balance 356.

FIG. 3H illustrates an additional embodiment of the viewing framework 302: an HCP dashboard 370. The HCP may view all of their current patients 366 in a single viewing framework 302 in the HCP dashboard 370. The HCP dashboard 370 may show patients' medical record numbers and date of birth, among others. The HCP dashboard 370 may also have complication summaries 368 for each patient, showing current risk assessments (i.e., risk of developing a condition or complication). Patients may be categorized with high, moderate or low risk based on their respective risk factors. The complication summaries 368 may be arranged based on a descending order of risk. In some embodiments, the complication summaries 368 appear in a truncated view with an option to expand to a full viewing framework listing all other possible complications and their associated risks. The date at which each of the patient's surgeries are scheduled to occur may be shown on the HCP dashboard 370. Alternatively, the data at which each of the complication summaries 368 is updated per patient may also be shown on the HCP dashboard 370. In addition, the HCP dashboard 370 may also display one or more blocks of current patients 366 receiving in-patient care and/or out-patient care (clinic care) for each HCP. The aforementioned features enable the HCP to observe, at a glance, which of their patients require immediate care, and helps the HCP to prioritize their rounds. Each block of current patients 366 is interactive and is used to navigate to a different embodiment of the viewing framework showing more specific details of a selected patient. The HCP may further create a new patient record through the viewing framework 302 by entering patient information or scanning a barcode associated with the new patient. The viewing framework 302 may be viewed through different platforms such as a web browser or a mobile application.

In some embodiments, the HCP dashboard 370 may comprise a navigation bar comprising a patient triage element, pending patients element and add patients element. The patient triage element may display the number of patients that are experiencing a high risk of medical complications. The pending patients element may display the number of patients that require their data to be uploaded from EHRs and a corresponding status (e.g., ready for review, percentage of EHR data retrieval, time remaining to retrieve data, interrupted retrieval). The add patients element leads a user to a patient onboarding process. The add patients element may further display an onboarding progress indicator and patient icon. The patient triage element may further display patient details including, but not limited to, a medical record number (MRN), name, age, sex, date of birth, surgery date, postoperative day number, operative details, medications, and circle of care. Medication information may comprise medication names, dosage, method of administration, order date, frequency, start and end date.

FIG. 3I illustrates an alternative embodiment of the viewing framework 302 in a preoperative mode. Viewing framework 302 summarizes all relevant items that an HCP will need to review in a clinical setting. The viewing framework 302 comprises a collapsible panel 374 having patient information 376, patient details 378 and post-operative outcome predictions 380. The patient information 376 comprising a patient's name, medical provider, primary diagnosis, sex, date of birth (DOB), age and allergies may be displayed and fixed within the viewing framework 302. The patient details 378 may comprise one or more categories such as problem and surgical history, operative details, radiology and medication history. When the one or more categories of the patient details 378 is selected by a user, a pop-up window appears, detailing the information for the respective category (refer to FIG. 3J as an example). The patient details may comprise dated information and timeline of the patient's medical history. For the medication history, each medication may be associated with a symbol with a specific colour indicating whether the particular medication is active for the current day. The post-operative outcome predictions 380 may comprise one or more categories of complications, wherein the one or more categories of complications may comprise respiratory complication, sepsis, bleeding complication, neurological complication, gastrointestinal complication, cardiovascular complication, urinary and renal complication, thrombotic complication, wound complication and other complications. Each of the one or more categories of complications may further comprise an indication of a level of risk (word and colour) and risk probability in percentage. For example, 0-33% may indicate low risk shown in green or grey, 34-67% may indicate moderate risk shown in orange, while 68-100% may indicate high risk shown in red.

The viewing framework 302 may further comprise a visual representation of a patient's body 382 with a date selector 384. The date selector 384 controls the information shown on the viewing framework 302. Based on the selected date, the HCP may be shown the viewing framework 302 in a preoperative mode or a post-operative mode. The selected date may also affect the information shown on timelines relating to patient details 378 within the viewing framework. In some embodiments, the date selector 384 may only show dates associated with obtained patient data, excluding days in between the days of relevance.

Similarly to FIG. 3A-3G, the patient's vitals and lab tests may be displayed on the viewing framework 302 with an associated name, icon, and gauge for each biomarker. Measurements for a current time period are mainly highlighted in the viewing framework 302, but changes in biomarker values compared to a previous time period may also be indicated. The measurements of the patient's vitals and lab tests may be further color-coded based on the current value it is within. For instance, normal, abnormal and critically abnormal measurements may appear to be green, orange and red, respectively. In some embodiments, the patient's vitals and lab tests in the viewing framework may be further expanded to show other metrics not in view. In the preoperative mode of the viewing framework 302, additional sections comprising a review of systems, a care plan and past visits may also be displayed.

The viewing framework 302 may also be presented in a postoperative mode. The viewing framework 302 generally comprises the same components as that of the viewing framework in the preoperative mode, displaying a collapsible panel, vitals, lab tests and a care plan. However, some components may be removed such as the review of systems and past visits. Additional components that may be found in the post-operative mode of the viewing framework 302 may comprise an input/output section and a physical assessment section. The input/output section displays biomarkers measured from fluid going through a sensor device within a monitoring system. The physical assessment section may comprise an abdominal assessment, nutritional status, hemodynamic and neurological assessment, and wound assessment. The post-operative outcome predictions within the collapsible panel may be updated daily as more data becomes available. By displaying all the aforementioned information, HCPs would easily navigate between post-operative days to get a quick assessment of patient recovery and overall progression.

Users of the viewing framework 302 may comprise one or more lab tests that a physician might want to evaluate for a given patient. The viewing framework is configured to have a selection of a plurality of lab tests that a user can select from. However, given the number of lab test options, lab tests that have abnormal results are prioritized to visible on the main screen of the viewing framework 302 in both the preoperative and post-operative mode. From the main screen, the lab test section may be expanded to view other lab test results and other lab test options in a lab test screen. The lab test screen may be split into different categories of lab tests, wherein the different categories may comprise general hematology, blood gas, chemistry profile/complete metabolic panel, liver function tests and coagulation profile, renal function tests, pulmonary function tests, and fluid balance. Other tests may comprise hematology tests, blood chemistry tests, drain amylase tests, endocrine function tests, microbiology and infectious disease tests, virology tests, bacteriology tests, mycology tests, parasitology tests, immunology and serology tests, oncology tests, and functional tests.

Components in the general hematology category may comprise hemoglobin (Hgb), leukocytes (WBC), erythrocytes (RBC), hematocrit (Hct), mean corpuscular volume (MCV), mean corpuscular hemoglobin (MCH), read blood cell distribution width, mean platelet volume, platelet count, neutrophils, lymphocytes, monocytes, eosinophils, basophils. The blood gas category may comprise venous and arterial pH, pCO2, pO2, HCO3, and O2 saturation. The chemistry profile/complete metabolic panel category may comprise sodium, potassium, bicarbonate, chloride, calcium, ionized calcium, magnesium, phosphate, iron, serum glucose, c-reactive protein, procalcitonin, amylase, lipase, vitamin B12 and ferritin. Liver function tests may comprise prothrombin time, fibrinogen, D-dimer, albumin, international normalized ratio, partial thromboplastin time, alanine aminotransferase, alkaline phosphatase, aspartate aminotransferase, lactate dehydrogenase and bilirubin. Renal function tests may comprise creatine, blood urea nitrogen and eGFR. Pulmonary function tests may comprise forced vital capacity (FVC), forced expiratory volume (FEV) and the ratio between FEV1/FVC. Lab tests displayed on the viewing framework are not only limited to the aforementioned components.

FIG. 3J illustrates a list of previous medical problems and surgical history of a patient in a viewing framework 302. In some embodiments, radiology test results, medication history and operative details may also be displayed in a pop-up window 386. The list of medical previous medical problems, surgical history, radiology test results, medication history and operative details comprise corresponding dates to inform HCPs of a timeline of events. These elements may be updated regularly, especially during a postoperative monitoring period to provide HCPs with sufficient details to determine whether the patient is experiencing any potential complications.

FIG. 3K and FIG. 3L illustrate customizable fields in a viewing framework 302. The customizable fields comprise an index or reference surgery 388, operative details 390, custom thresholds 392 and a patient's circle of care 394. EHR systems allow institutions to set thresholds for lab results and vitals. Using these existing systems, HCPs typically receive notifications whenever a value exceeds a set threshold, often resulting in excessive alerts. The disclosed systems and methods comprise contextualizing the plurality of different information types involves taking into account individual patient baselines which may be impacted by comorbidities and risk factors, allowing for more accurate, relevant and personalized alerts and preventing notification fatigue. HCPs may set up custom thresholds for lab results and vitals their patients during onboarding or at later stages, which allow other members of the circle of care to know exactly when patient care should be escalated. The custom thresholds may comprise a critical low value, critical high value and the corresponding unit for each lab result or vital element.

Patient onboarding is streamlined through automatic EHR integration that pre-populates data, minimizing manual input and reducing the risk of errors. To initiate the onboarding process, HCPs may input the medical record number (MRN) of a patient into the disclosed system to retrieve patient data via EHR integration. If patient data is found, HCPs are prompted to confirm the pre-populated information. Otherwise, HCPs may be prompted to re-enter the MRN or add a manual entry instead. In some embodiments, a plurality of patients may be onboarded at the same time and an indicator may be present to show the status of the EHR data retrieval. For patients that have undergone surgery, HCPs may select or input an index surgery or reference surgery. The index surgery is required to select the correct input for the risk calculators. Once the index surgery is selected, additional operative details that could not have been pulled from EHR may be entered. The additional operative details may comprise surgery and procedure details (e.g., type of surgical procedure, start and end date and time, lead surgeon, blood transfusion status, ASA score), preoperative risk factors (e.g., demographics, medical history, surgical history, family history), intraoperative risk factors (e.g., organ texture and size, anesthetic management, hemodynamic factors) and postoperative status and disposition. An embodiment of the viewing framework displaying the additional operative details is shown in FIG. 3K. In addition, custom patient thresholds for lab tests and vitals may be entered, allowing for clinical context to be considered as shown in FIG. 3L. The patient's circle of care is also entered during onboarding from EHR or manual entry to determine each HCP involved in taking care of the patient (e.g., primary care physician, fellow or resident, ward physician, ICU physician, other specialists, etc.). Other inputs to the patient's circle of care may include an inpatient location and bed assignment.

HCPs may prioritize high risk patients through a triage feature on the displayed dashboard. HCPs may monitor the lab test results, vitals, inputs, outputs, as well as access operative details through the patient view, wherein abnormal values are clearly indicated through color coding. HCPs may review different biological systems of the patient through the system view. In the system view, the risk of different complications associated according to each biological system is shown. In some embodiments, existing risk calculators may be used and a selection of the existing risk calculators may be available to HCPs. HCPs may view the risk calculator inputs obtained via EHR or manually edit the entries if necessary. Additionally, HCPs may review the source of the risk calculators, risk factors, signs and symptoms, diagnostic reports, lab test results, and medications associated with each biological system.

FIG. 4 illustrates a mobile display framework 402 of a viewing framework 302. The mobile display framework 402 may display all of the elements described in FIG. 3A-3L. The mobile display framework 402 may further send notifications to the HCP to alert them to ongoing updates 408 and/or emergencies with their patients.

The mobile display framework 402 may display summaries 404 (summarizing the number of patients who are at high risk, needing further assessment, or are stable, for example). The mobile display framework 402 may display patient status 406 for one or more patients. The patient status 406 may be sorted in terms of importance. For example, the most critical patients may be found at the top. The healthcare practitioner may further be able to view summaries 404 pertaining to their patients 410, or to all of the patients being treated by the entire department 412.

FIG. 5 illustrates an alternate embodiment of a viewing framework 502. The alternate viewing framework 502 illustrates a visual representation of a patient's body 512 in 2D form. In some embodiments, the visual representation of the patient's body 512 may be illustrated in 3D form. The visual representation 512 may show the localization of symptoms present, allowing an HCP to assess the patient's condition at a glance. The alternate viewing framework 502 may further comprise one or more gauges 504 indicating a risk level of the patient experiencing one or more medical conditions that may be a complication from a surgical procedure (e.g., pulmonary embolism, acute mesenteric ischemia, urinary tract infection (UTI), bronchitis/pharyngitis, etc.). Another portion of the alternate viewing framework 502 may also comprise subsections 508, each pertaining to different groups of biological systems (e.g., head, eyes, ears, nose and throat (HEENT), pulmonary, abdominal, etc.) and one or more symptoms experienced by the patient in each of the groups of biological systems.

Regardless of the viewing frameworks 302, 502, the system organizes a viewing framework based on a back-end sub-portfolio stored in a memory of the system that includes patient-related data that may be derived from EHR data (e.g., diagnosis, diagnostic reports, progress notes, procedures, vital signs, lab tests, ADT, medications). The viewing framework displays symptomology and other clinical features such as comorbidities and risks pertaining to each biological system of a patient. Different symptoms may be processed and extracted from free-form medical text in EMR with LLMs to accurately extract keywords and summaries to describe each biological system status, wherein each biological system status is displayed under each biological system within the viewing framework. The viewing framework also displays post-operative risk predictions for possible adverse events following a surgical procedure. One or more risk levels are indicative of a likelihood of adversity. In some embodiments, the one or more risk levels are scored as a percentage and thresholds of severity are color-coded (e.g., red: high; yellow: medium; green: low). The post-operative risk predictions may require processing of structured or semi-structured clinical data with traditional machine learning models to accurately predict risk levels of adverse events.

The viewing framework may further comprise a human body visualization of the patient through a 2D or 3D human model 512, displaying symptom localization on the human model to access the patient's condition at a glance. Pain levels 506 experienced by the patient may also be extracted from EMR. Users of the viewing framework may also dynamically assess the patient's condition over time. In some embodiments, custom alerts may also be created using IFTTT when the patient is monitored for certain parameters (e.g., hemodynamics, sepsis, venous thromboembolism). One or more parameters may be selected by the patient's HCP that allows the viewing framework to display a filter view to only relevant patient parameters (e.g., risk factors, biomarkers, scans, etc.). The one or more parameters may also be highlighted according to custom criteria.

In addition, the viewing framework may identify one or more surgical sites 510 for HCPs to determine proper wound care based on the classification of the one or more surgical sites based on depth, cleanliness, etc. Clinical guidelines (e.g., ERAS guidelines) may also be integrated within the viewing framework to match them against patients and provide real-time recommendations accordingly. Images and annotations of radiological scans may also be shown on the viewing the framework. In some embodiments, the viewing framework may require PACS integration.

The viewing framework may log all transactions between a plurality of systems, medical records, and users accessing the medical records. In some embodiments, the viewing parameter may comprise a “cruise” feature to highlight symptoms, signs, lab results and vitals from EMR and associate them with the overall biological system that each one is associated with (e.g., creatinine: renal; RUQ pain: renal, etc.). The “cruise” feature allows HCPs to be supported with making a differential diagnosis based on both ROS and labs/vitals.

FIG. 6 illustrates the architecture for an extensive EHR (and other medical data) may be integrated into a single, centralized system for streamlining patient care and facilitating inter-hospital collaboration and clinical study development. In order to integrate hospital-acquired data such as EHRs, the systems and methods disclosed herein are an enabler for pulling, processing, and displaying, the required non-device data for displaying visuals, insights, risk modelling, analytics and dashboarding.

EHRs may be obtained from one or more sites 614, databases or platforms. The derived EHR data is integrated into the centralized system via an extraction subsystem 606 wherein the centralized system may comprise an integration engine 610 in an on-premise server for EHR integration 602. The integration engine 610 is used to manage connections with the one or more sites 614. The integration engine processes raw EHR data and different standards such as L7 v2 and FHIR into JSON format before sending the data to a queue, wherein the queue is a first in first out queuing service. In some embodiments, the queue may be part of the integration engine. In some embodiments, raw EHR data is stored in a raw local storage 618 comprising a first set of a plurality of schemas for observations, medications, procedures, etc. The schemas may be expanded to accommodate additional fields. A data transformation engine 608 is then used to transform raw EHR data into processed EHR data, and poll the raw data in regular time intervals. Once the EHR data is processed, the processed EHR data is stored in an on-premise processed local storage 620 comprising a second set of a plurality of schemas for sites and devices, among others. Under an application communication engine 622, the processed EHR data is exposed to application programming interfaces (APIs) with GET and POST endpoints to service different platforms and applications. The integration engine, data transformation engine, and application communication engine, raw local storage service and processed local storage service may be run in a Docker container 616. Different platforms such as the Stream™ application 624, web portal 626 or mobile application 628 may then be used to view the processed EHR data. System logs and anonymized EHR data may be stored in a processed cloud storage 630 and further analyzed in a data warehouse 632. Other embodiments of the architecture of the system may comprise involving a processed cloud storage instead of an on-premise processed local storage, excluding a data warehouse, having an API gateway, and integrating large language models in different applications.

Data obtained from EHRs may comprise (specific to a patient), but are not limited to: medical record numbers (MRN), patient names, demographics, procedures (surgical procedures, anesthesiology summaries, other relevant non-surgical procedures), vital signs, lab tests, ADT (including discharge summaries), diagnosis, medications, diagnostic reports (radiology, X-ray, CT, ultrasound), clinical notes (progress notes, nurses notes, physician summaries, assessment reports), and audit Logs (readmissions, ICU admissions, mortality, procedure logs and time). Additional data needs from Picture Archiving and Communication Systems (PACS) systems will be required to pull medical imaging (for radiology, X-ray, CT, ultrasound).

The EHR system used at a given hospital preferably supports the following standards: HL7 v2.x FHIR Integration Engine to process raw EHR data, various integration engines may be employed (alone or in combination with other integration engines). An example of an integration engine known in the art may comprise Mirth Connect.

According to an aspect of the disclosure, there is provided: a health information system for visualizing patient health information data, the system comprising: one or more subsystems adapted to receive health information data relating to one or more patients' health conditions or to each of the one or more subsystems' statuses; a computer system communicatively coupled to a memory, a processing unit, and to each of the one or more subsystems, the server continuously receiving the measured data from at least one of the one or more subsystems; a display device, communicatively coupled to the server, the display device configured to display a viewing framework; wherein the processing unit is further configured to detect, via a viewing framework decision engine, viewing parameters, the viewing parameters comprising one or more of: a display device type, a user's credentials, and a location of the display device, and the processing unit is further configured to: determine, based on the viewing parameters, the viewing framework; and automatically send the viewing framework to the display device for displaying the viewing framework.

In some embodiments, the viewing framework further performs one or more of the following functions: update and display a risk assessment related to the one or more patients, sort risk assessment based on level of risk, and alert a healthcare practitioner that a patient is in need of immediate care. In some embodiments, the risk assessment is based on one or more health thresholds relating to a condition. In some embodiments, the one or more health thresholds are updated based on a location of the patient. In some embodiments, the viewing framework displays one or more patient parameters relating to: vitals, medications, tests, pre-operative data, fluid data, the one or more patient parameters being specific to a patient. In some embodiments, the viewing framework is adaptable to one or more user inputs, the one or more user inputs specifying which of the one or more patient parameters to display. In some embodiments, the viewing framework is adaptable to one or more user inputs, the one or more user inputs specifying which of the one or more patient parameters to display.

In some embodiments, the alert is a visual alert displayed on the display device.

In some embodiments, the alert is a voice alert played aloud on the display device. In some embodiments, the alert is communicated to the healthcare practitioner at set milestones post-surgery. In some embodiments, the alert is communicated to the healthcare professional at milestones automatically determined by a machine learning engine, the milestones relating to optimal times or conditions at which a risk assessment should be measured. In some embodiments, the alert comprises a health report displaying data that is tailored to one or more of: patient subgroup, healthcare professional, and hospital department. In some embodiments, the alert is communicated to the healthcare professional at milestones automatically determined by a machine learning engine, the milestones relating to optimal times or conditions at which a risk assessment should be measured.

In some embodiments, the risk assessment comprises an interactive gauge displaying current values, the interactive gauge having a one-click function. In some embodiments, the one-click function comprises flipping the interactive gauge to a trendline showing a trend in the values. In some embodiments, the viewing framework further comprises displaying one or more prompts for: measuring vitals, taking tests, and wherein the prompts are updated and displayed based on a user's condition. In some embodiments, the location of the display device comprises one or more of: at a point of care and remote from the point of care. In some embodiments, the display device at a point remote from the point of care is a mobile device. In some embodiments, wherein the display device at a point remote from the point of care is a desktop device. In some embodiments, wherein the one or more subsystems comprise one or more of: medical devices, inline medical devices, electronic records systems, clinical notes, clinical research, subjective observations, progress notes, sensor data, patient billing data.

In some embodiments, there is provided: a computer implemented health information method for visualizing health information data, the computer implemented method comprising: receive, at one or more subsystems, data relating to one or more patients' health conditions or to each of the one or more subsystems' statuses; receive, at a computer system communicatively coupled to a memory, a processing unit, and the one or more medical devices, the data from at least one of the one or more subsystems; display, on a display device communicatively coupled to the server, a viewing framework; wherein the processing unit is further configured to detect, via a viewing framework decision engine, viewing parameters, the viewing parameters comprising one or more of: a display device type, a user's credentials, and a location of the display device, and the processing unit is further configured to: determine, based on the viewing parameters, the viewing framework; and automatically send the viewing framework to the display device for displaying the viewing framework.

According to an aspect of the disclosure, there is provided: a non-transitory computer readable storage medium for health information visualization, the non-transitory computer readable storage medium comprising: a processor; a memory, the memory comprising instructions that, when executed, configure the processor to: receive, data from at least one of one or more subsystems receiving data relating to one or more patients' health conditions or to each of the one or more subsystems' statuses; display, on a display device communicatively coupled to the server, a viewing framework; wherein the processing unit is further configured to detect, via a viewing framework decision engine, viewing parameters, the viewing parameters comprising one or more of: a display device type, a user's credentials, and a location of the display device, and the processing unit is further configured to: determine, based on the viewing parameters, the viewing framework; and automatically send the viewing framework to the display device for displaying the viewing framework.

According to an aspect of the disclosure, there may be provided a centralized health information management system 604 for managing health information data, the system comprising: one or more sites 614 or databases or platforms; an extraction subsystem 606 for extracting health information data from one or more platforms; and a computer system 602 communicatively coupled to a memory, a processing unit, and to the extraction subsystem, the computer system 602 adapted to receive the extracted health information data from the extraction subsystem; wherein the processing unit further comprises: a consolidation engine 610 adapted to automatically convert extracted data into usable data; a decision engine 608 adapted to detect context information associated with the usable data, and, based on the context information, automatically send each of the usable data to one or more of: an evaluation system, a clinical study, a clinical study model, and a system integration system.

In some embodiments, the one or more platforms comprise one or more of: medical devices, inline medical devices, electronic records systems, clinical notes, clinical research, subjective observations, progress notes, sensor data, patient billing data.

In some embodiments, the extraction subsystem comprises one or more of: optical character recognition, voice-to-text, and automatic extraction of digital data over a network.

In some embodiments, the decision engine comprises one or more of: an AI engine or a machine learning engine.

In some embodiments, the centralized health information data management system further comprises a compatibility component configured to adapt the computer system to interface with a plurality of hospital IT systems.

In some embodiments, the centralized health information data management system further comprises a security component, the security component comprising one or more of: data encryption, password protection, data anonymization, and two-factor authentication.

In some embodiments, the evaluation system is adapted to detect and evaluate one or more trends in one or more of the usable data.

In some embodiments, the evaluation system is further adapted to develop a risk assessment relating to a health condition based on the one or more trends.

In some embodiments, the evaluation system is further adapted to detect and alert to similar cases based on the one or more trends in the usable data.

In some embodiments, the evaluation system is adapted to determine one or more diagnoses for one or more patients, and wherein each diagnosis of the one or more diagnoses is coupled with a treatment plan, the treatment plan being extracted by the extraction subsystem from at least one database of a plurality of databases.

According to an aspect of the disclosure, there may be provided: a computer implemented centralized health information data management method for managing health information data, the computer implemented method comprising: extracting, via an extraction subsystem for health information data from one or more platforms; and receiving, at a computer system communicatively coupled to a memory, a processing unit, and to the extraction subsystem, the extracted health information data from the extraction subsystem; automatically converting, via a consolidation engine of the processing unit, the extracted data into usable data; detecting, via a decision engine of the processing unit, context information associated with the usable data; and based on the context information, automatically sending each of the usable data to one or more of: an evaluation system, a clinical study, a clinical study model, and a system integration system.

According to an aspect of the disclosure, there may be provided a non-transitory computer readable storage medium for centralized health information data management, the non-transitory computer readable storage medium comprising: a processor; a memory, the memory comprising instructions that, when executed, configure the processor to: extract, via an extraction subsystem for health information data from one or more platforms; and receive, at a computer system communicatively coupled to a memory, a processing unit, and to the extraction subsystem, the extracted health information data from the extraction subsystem; automatically convert, via an consolidation engine of the processor, the extracted data into usable data; detect, via a decision engine of the processor, context information associated with the usable data; and based on the context information, automatically send each of the usable data to one or more of: an evaluation system, a clinical study, a clinical study model, and a system integration system.

The evaluation system may be adapted to detect and evaluate one or more trends in one or more of the usable data (and evaluate risk to a user based on the one or more trends).

The decision engine may be adapted to, automatically, upon receiving an input having data, flagging similar cases/trends, and automatically determining a treatment plan based on the risk and available guidelines to the hospital system. The decision engine may work off of a database having data from a plurality of sources, and the data may be filterable by one or more of the following criteria: surgeon, hospital, region, country.

The memory may comprise an integrated database of patient data from a plurality of hospitals/clinics, and the decision engine may be adapted to develop clinical studies, using the integrated database and one or more user inputs, which present the user with factors required to complete a clinical study.

The health information data management system 604 may further have one or more security components in order to manage user access to the database.

The health information data management system 604 may further comprise one or more algorithms configured to process data with missing values, comprising: deciding when receiving data, how best to correct the missing values, based on the patient population and/or the intended recipient of an output of data, and/or which type of data is missing.

The data management system's extraction subsystem may extract structured or unstructured data from a variety of sources (for example, clinical notes) to develop a report, displayable on a display element. The report may be displayed at regular intervals. For example, pain analysis, which is highly subjective, can be used to train a ML model by extracting it, highlighting it with various flags or other mechanisms, entered into a ML model, in order to specifically improve subjective observations, and from multiple people, in order to catch complications earlier.

The data management system may be further adapted to develop indication-specific models based on processed clinic/clinician data: The system may produce either predictive models patient status models, based on any one of the following data extracted by the extraction subsystem 606: patient's billing information (which might include limited but easily accessible clinical notes), billing info/codes, to map clinical procedures, diagnoses, conditions, sensor data, medical device data, etc.

ML algorithms may comprise and employ fuzzy logic in order to chart a patient journey. The models may be trained with probable pathways (detecting early indications of complications and confirming later complications). ML algorithms may be employed to fill in missing data, or process data accurately with missing values, and test models with limited datasets. The ML model may also be employed to detect improvements in suggested procedures to ensure consistency across multiple hospitals, and to flag non-compliance or non-uniformity in suggested procedures, and evaluate trends in regular, uncaptured data, including daily rounds, progress notes etc. Integrating a plurality of data may enable HCPs to employ the system to develop post-surgical guidelines and more sensitive flagging methods. The system may automatically extract EHR data to be sent to a clinical study for processing.

According to an aspect of the disclosure, there may be provided a health information portal system for managing patient care, the health information portal comprising: one or more databases coupled to a memory, the one or more databases having a plurality of patient data, the patient data extracted from one or more subsystems; a processor coupled to the memory, the processor configured to: receive an input from one or more users, the input comprising one or more of: queries, data, and notes; detect, via a decision engine of the processor, context information associated with the input; based on the context information, couple the input with an output comprising one or more of: related data from the one or more databases, contact information associated with a manager of the patient data, treatment plans, indication specific models; and display the output for the one or more users on a display element; an anonymization component configured to anonymize the plurality of patient data; and an integration component for integrating the health information portal system with each hospital IT system of a plurality of hospital IT systems.

In some embodiments, the one or more subsystems comprise one or more of: medical devices, inline medical devices, electronic records systems, clinical notes, clinical research, subjective observations, progress notes, sensor data, patient billing data.

In some embodiments, the input further comprises a query from the user relating to a means of summarizing the data.

In some embodiments, the query is processed by one or more of: an AI engine or a machine learning engine, and the output comprises one or more of: a patient specific report, a department-specific report, a user specific report, and a summary of relevant cases.

In some embodiments, the output further comprises displaying one or more actionable outputs relating to: doctor-specific recommendations, hospital-specific recommendations, country-specific recommendations, and demographic specific recommendations, the actionable outputs comprising recommendations for improving one or more outcomes.

According to an aspect of the disclosure, there may be provided a computer implemented method for providing a health information portal, the computer implemented method comprising: receiving, at a memory having one or more databases, a plurality of patient data, the patient data extracted from one or more subsystems; at a processor coupled to the memory: anonymizing the plurality of patient data with an anonymization component of the processor; integrating, with an integration component of the processor, the health information portal with each hospital IT system of a plurality of hospital IT systems; receiving an input from one or more users, the input comprising one or more of: queries, data, and notes; detecting, via a decision engine of the processor, context information associated with the input; coupling, based on the context information, the input with an output comprising one or more of: related data from the one or more databases, contact information associated with a manager of the patient data, treatment plans, indication specific models; and displaying the output for the one or more users on a display element.

According to an aspect of the disclosure, there may be provided a non-transitory computer readable medium for providing a health information portal, the non-transitory computer readable medium comprising: a processor; a memory, the memory comprising instructions that, when executed, configure the processor to: receive from the memory, a plurality of patient data, the patient data extracted from one or more subsystems; anonymize the plurality of patient data with an anonymization component of the processor; integrate, with an integration component of the processor, the health information portal with each hospital IT system of a plurality of hospital IT systems; receive an input from one or more users, the input comprising one or more of: queries, data, and notes; detect, via a decision engine of the processor, context information associated with the input; coupling, based on the context information, the input with an output comprising one or more of: related data from the one or more databases, contact information associated with a manager of the patient data, treatment plans, indication specific models; and display the output for the one or more users on a display element.

The portal may be coupled to the centralized health information management system 604 in order to extract a plurality of data. A plurality of doctors may be able to access the portal and enter data relating to their cases for evaluation by the portal. The portal may flag similar cases based on worldwide database of patient data, suggest treatment plans based on identified similarities, and facilitate connection and communication with professionals having experience with a similar case or relevant research) on demand or automatic. Communication systems such as video calls or chat boxes may be integrated into the portal.

The portal may be equipped to develop indication-specific models by extracting and combining literature, guidelines, new classification methods, biosignal trends, etc. Based on questionnaire, the portal may provide a user with a clinical study design, with a predicted required sample size, what data is required (biomarkers), financial commitment, etc.

The viewing framework 706 may be coupled with a plurality of back-end, hospital management systems, described and illustrated in FIG. 7A-FIG. 7B. Hospital management systems may be integrated with the viewing framework(s) and/or their linked databases. The hospital management systems may be linked to the viewing framework(s) to allow the user to manage their clinical workflow between surgery, rounds, and clinic and access other portfolio features from the convenience of their mobile device.

One hospital management system is the exemplary skills management system 708, which tracks hospital metrics 702, and identifies areas for improvement, as well as areas where the hospital is at risk. Hospital metrics 702 may comprise procedures performed, average surgery times, readmissions, and the like. They may be tracked as departmental averages 710, and patient by patient 712, such that managers may view how a given patient is receiving treatment. The hospital metrics 702 may be sorted by surgeon, specialty, demographics, and the like.

Based on EHR data relating to one or more patients, procedures, diagnoses and audit logs, surgeon insights and departmental insights may be displayed in the hospital management system. Surgeon insights and departmental insights may comprise analytics on surgeries performed, patient cohorts, complications, readmissions, ICU admissions, procedure duration, among others. In order to display these insights within the hospital management system, data warehousing is required to process and prepare data for analytics, wherein the analytics are processed or performed on structured data in the data warehouse to produce relevant insights. Business intelligence (BI) tools are then used to connect to the data warehouse and display the insights.

Hospital management systems may further comprise one or more data management systems, integrated with the viewing framework. A remote management system may allow a user to manage their clinical workflow between surgery, rounds, and clinic while accessing other data displayed by the viewing framework. A doctor may view their upcoming procedures.

A manager or assistant may view the department's upcoming procedures and may schedule new ones accordingly in an exemplary data management system. The data management system may comprise in-patient schedules (ward, ICU), procedure schedules, clinic schedules and a virtual chatbot (e.g., ROSA™). In-patient schedules, determined by ADT data processing of triaged patients linked with an associated risk, are set to highlight and rank patients according to risk to prompt more guided rounds (i.e., critical patients need more time and attention). Procedure schedules are configured to allow filtration and grouping by surgeon, department and date, as well as procedure details such as location, attending surgeons, nursing staff, pre-operative diagnosis, and radiological scans, among others. Clinic schedules may be configured in a separate web portal application. The virtual chatbot may be used to generate an oral and/or written endorsement report on all patients, especially high-risk patients, highlighting findings from surgical and/or nursing progress notes. The virtual chatbot may further process text inputs and audio inputs to generate real-time patient updates and endorsements to HCPs. In addition, the virtual chatbot may alert (e.g. initiate a call) HCPs if a patient has clear signs of deterioration and verbally communicate hospital guidelines to HCPs.

Furthermore, a report management and integration system may process unstructured free-form medical texts with large language models (LLMs). As such, the report management and integration system convert a plurality of medical data and information into usable, relevant information that can guide patient care. The transformed, usable and relevant medical information may be in the form of one or more reports.

Reports generated by one or more data management systems disclosed herein may comprise custom reports, discharge reports, operative reports, and consultation reports. The custom reports may be automated wherein the reports summarize and highlight key patient journey insights from EHR data comprising admission insights, diagnosis and comorbidities, significant events (ICU admission, surgical procedures, etc.), risk levels, and patient discharge summaries based on EHR data including procedures, vital signs, lab tests, admission, discharge and transfer (ADT) data, diagnosis, medications, diagnostic reports and progress notes. The EHR data may be processed depending on its structure. Unstructured free-form medical texts may be processed with LLMs to accurately extract key words and summaries to provide qualitative insights. Structured/semi-structured clinical data may be processed with traditional machine learning models to accurately predict risk levels of adverse events. Structured/semi-structured clinical data may be processed with business logic to highlight key events.

The discharge reports may be automatically generated from ADT data. The operative reports (short-term or long-term) and the consultation reports may be generated by processing audio and video data with LLMs to transcribe and summarize them into text. The short-term operative reports may transcribe and summarize the operating room data after surgery just as a surgeon would have written it from memory. In a similar manner, the consultation reports may transcribe a patient and consultant interaction during a clinic visit. Conversely, the long-term operative reports may ingest audio and video from surgery and automatically generate a report that expands on the short-term report accordingly.

Machine learning models may be trained or re-trained for improved accuracy over time. Risk factors and medical complications experienced by a patient may be used as new training data for the machine learning models to improve how complication risk predictions are determined within the system. The machine learning models may further use acquired patient data as training data to provide more accurate and relevant clinical recommendations. Clinical recommendations may be derived from a plurality of professional medical organizations, public health institutes, and evidence-based medical publications. In some embodiments, clinical recommendations may be derived from a custom machine learning model. In other embodiments, clinical recommendations may be derived from existing point of care tools, which are integrated within the disclosed system.

Processing large amounts of raw patient data from various sources to produce clear consolidated patient data and important insights may be infeasible for humans to mentally process in a timely and accurate manner. This involves extracting, filtering, consolidating and converting the raw patient data into usable and interpretable data. Given the vast number of responsibilities HCPs have, the machine learning techniques integrated within the disclosed system allow for critical information to be readily apparent for HCPs to make effective and efficient clinical decisions. The medically sound clinical decisions may comprise ordering additional lab tests, prescribing medications, suggesting other interventions (e.g., surgery, optimized treatment plans), referring patients to other specialists, determining a discharge date and/or moving patients to a more suitable department within a given healthcare institute or to another healthcare institute.

In some embodiments, the solutions architecture may be an on-premise solution or a hybrid solution. The infrastructure of the hybrid solution may comprise having a subsystem information consolidation engine run on a dedicated physical or virtual machine within a given hospital's network. LLMs may require a dedicated host on a virtual machine in the cloud with sufficient memory and processing capability depending on the LLM parameter size. Some processing applications may run on standard virtual machines in the cloud while others may run on the hospital's physical or virtual machine. Data warehouses may run on the cloud using relevant services. Furthermore, network, security and privacy of the hybrid solution may comprise having the hospital enable EHR access for the subsystem information consolidation engine to pull data via TCP or HTTPS. The processed data may be accessed through TLS encryption. The hospital may provide an exposed port network for cloud connectivity which can be accessed by VPN. The hospital may also configure its network firewall to allow for external network access. In the cloud, EHR data is anonymized with all PHI de-identified following HIPAA standards. The on-premise solution mostly has the same features as that of the hybrid solution. The differences in infrastructure include LLMs requiring a dedicated host on a physical or virtual machine and running all processing applications on a standard physical or virtual machine within the hospital's network. As for network, security and privacy, hospitals may allow for VPN or SSH tunneling to remotely access the locally hosted machines for troubleshooting and maintenance. For the on-premise solution, EHR data remains within the hospital's network.

The skills management system 708 may also track individual doctor metrics 704 (or any HCP) for evaluating their performance, as shown in FIG. 7B. The doctor metrics 704 may comprise procedure breakdowns, a total number of surgeries, complications, ICU admissions, and readmissions for one or more patients, The doctor metrics 704 may also comprise a risk index performance bar 714 which compares an overall patient risk level associated with a department, institution and a doctor. Further graphical representations of patient comorbidities 716 may also be included in the skills management system 708. The hospital management system may further comprise a searchable, and communicative database system for streamlining patient care. A healthcare provider may query, within the searchable database, patient records to export records of interest relating to their research. Cohort exports require processing the query requests and exporting the records of interest through a downloadable file such as an Excel and/or CSV file.

The database system would preferably redact all patient-identifying information. The database system would preferably generate a master record log which may be stored confidentially in order to restore and re-identify records, if they had the credentials to do so. The de-identification or anonymization feature is to protect patient privacy, especially as patient records are exported.

Preferably, the database system is connectable to multiple organizations. Surgeons, departments and hospitals within a healthcare system may share data across organizational lines in order to enable easy sharing and collaboration across organizations during research or during a difficult treatment.

Preferably, this database system is run by a large language model (LLM), which can be easily interacted with. For example, a surgeon may type their patient's symptoms into the database system, and the system may recommend literature, or patient records, or a surgeon from another hospital, that the first surgeon may consult, in order to develop a more educated framework for viewing their patient's condition. The database may be linked to a communication system, such as video or phone calling, or messaging, such that the surgeon may instantly communicate with HCPs from other organizations in order to research their patient's condition. The LLM may not only serve to pull patient information or literature from the database but may also act to analyze structured data and generate accurate plots, graphical representations and relevant statistics. The hospital management system allows for cohort exports, de-identification or anonymization, data sharing across organizational lines, and an interactive chatbot-like analysis and visualization.

FIG. 8 illustrates a diagram with a plurality of postoperative complications arranged according to different appropriate categories. The complication categories 802 are based on a different biological system (e.g., respiratory, gastrointestinal), wherein each category 802 may display an aggregated risk percentage or level based on each complication card 804 within each biological system. For example, under gastrointestinal complications, complication cards may comprise anastomotic leak and postoperative pancreatic fistula (POPF). If anastomotic leak and POPF are associated with moderate risk and low risk, respectively, the gastrointestinal complication category indicates an overall moderate risk. In some embodiments, multiple risk calculators may be accessed via each complication card. A default calculator may also be set in place according to an order of priority: a calculator with the highest support in evidence, a calculator with tier 1 data, a calculator with tier 2 data, and a calculator with tier 3 data. Tier 1 data may comprise structured data mandatory for implementation. The tier 1 data may further represent a minimum requirement that institutions must have readily available in EHRs. Tier 2 data may comprise low risk unstructured sources where low subjectivity and high retrieval accuracy are expected. Tier 3 data may comprise high risk unstructured sources, where subjectivity or inconsistency are expected.

The present disclosure describes systems and methods that identify and integrate individual risk factors, comorbidities, lab test results, symptoms, vitals and radiology test results to predict and detect early organ or biological system risks. Artificial intelligence is utilized to extract relevant information from a plurality of patient data, including free-text clinical notes, to summarize a patient's condition on the bedside. The disclosed system allows for HCPs to remotely access patient information through a web browser or mobile application. In terms of report management and integration, clinical reports may also be drafted automatically, summarizing the patient's medical history and present condition from disparate and unstructured sources. In some embodiments, different comparative dashboards may be provided to enhance operational efficiency for better healthcare quality and departmental performance.

A searchable and communicative database for streamlining patient care may be integrated to facilitate easier collaboration between researchers both within a given hospital and across institutions globally. EHRs are also integrated to pull and process data from multiple sources to enable the aforementioned features. In some embodiments, voice-based querying of the database may be integrated as well. This system automates analysis for clinical research, providing advanced research analytics that may comprise report generation with charts, graphs, and data insights. The system may further be used for problem solving using a custom generative pre-trained transformer (GPT). Furthermore, the disclosed system may involve on-premise or hybrid implementations. For on-premise implementations, the system with EHR integration comprises the use of on-premise networks only, without any PHI data permitted to leave the on-premise networks. For hybrid implementations, the system with EHR integration is based on a mixed on-premise and cloud-based model. The system is further configured to comply with a plurality of different security, storage and privacy standards following its development and implementation to ensure high quality and adherence to best practices.

The present disclosure describes systems and methods for managing health information data to deliver personalized care based on data-driven insights. These insights may be generated with data from a sensor device enabled to continuously monitor important patient parameters such as vital signs and biomarkers in drainage fluid, EHR data and novel algorithms to generate automated insights into the patients' risk profiles and recovery trends. LLMs are used to process unstructured data from EHR and convert it to a structured, usable format. Other machine learning models may also be integrated within a computer-based platform, wherein the machine learning models are trained from various patient records collected from global institutions, clinical databases and EHR data vendors. Having processed health information allows for the systems and methods to provide HCPs augmented clinical judgements and predictive insights that prompt them to make more effective clinical decisions.

In some embodiments, the machine learning models use a tree-based gradient boosting machine architecture. Other models may also be implemented in the system, wherein the models may comprise statistical models (e.g., logistical regression) and/or a custom rule system from publicly available studies. These machine learning models may be trained for different postoperative days to generate postoperative predictions. In some embodiments, training data for the machine learning models are processed, wherein processing the training data is completed by cohort development (e.g., data extraction, cohort definition, data validation and integration, quality assessment), and label development (e.g., clinical review, grade assignment, temporal annotations, multi-modal verification using clinical, radiological, and laboratory data, and quality assurance via multi-reviewer consensus). Model adaptation to a specific use case (e.g., feature mapping, model fine-tuning, performance validation) is then completed before the machine learning models are clinically implemented (e.g., alert system deployment, performance monitoring, clinical workflow integration, automated documentation). The machine learning models may be used to calculate risk probabilities.

Data disclosed herein may be in relation to preoperative data, intraoperative data or postoperative data. Taking into account different clinical guidelines and emerging research, the systems and methods allow for the generation of data visualization, clinical recommendations, and dynamic risk scores to be presented within the computer-based platform. Important data trends are highlighted to support better decision making, most up-to-date evidence is used to provide clinical recommendations, and continuous predictions are provided for several complications, forming a comprehensive platform for HCPs to more effectively care for their patients. HCPs may use a bedside monitor, mobile application, and/or web application to access the computer-based platform to manage their patients as well.

The present disclosure includes systems having processors to provide various functionality to process information, and to determine results based on inputs. Generally, the processing may be achieved with a combination of hardware and software elements. The hardware aspects may include combinations of operatively coupled hardware components including microprocessors, logical circuitry, communication/networking ports, digital filters, memory, or logical circuitry. The processors may be adapted to perform operations specified by a computer-executable code, which may be stored on a non-transitory computer readable medium.

The steps of the methods described herein may be achieved via an appropriate programmable processing device or an on-board field programmable gate array (FPGA) or digital signal processor (DSP), that executes software, or stored instructions. In general, physical processors and/or machines employed by embodiments of the present disclosure for any processing or evaluation may include one or more networked or non-networked general purpose computer systems, microprocessors, field programmable gate arrays (FPGA's), digital signal processors (DSP's), micro-controllers, and the like, programmed according to the teachings of the exemplary embodiments discussed above and appreciated by those skilled in the computer and software arts. Appropriate software can be readily prepared by programmers of ordinary skill based on the teachings of the exemplary embodiments, as is appreciated by those skilled in the software arts. In addition, the devices and subsystems of the exemplary embodiments can be implemented by the preparation of application-specific integrated circuits, as is appreciated by those skilled in the electrical arts. Thus, the exemplary embodiments are not limited to any specific combination of hardware circuitry and/or software.

Stored on any one or a combination of computer readable media or non-transitory computer readable media, the exemplary embodiments of the present invention may include software for controlling the devices and subsystems of the exemplary embodiments, for processing data and signals, for enabling the devices and subsystems of the exemplary embodiments to interact with a human user or the like. Such software can include, but is not limited to, device drivers, firmware, operating systems, development tools, applications software, and the like. Such computer-readable media further can include the computer program product of an embodiment of the present invention for preforming all or a portion (if processing is distributed) of the processing performed in implementations. Computer code devices of the exemplary embodiments of the present invention can include any suitable interpretable or executable code mechanism, including but not limited to scripts, interpretable programs, dynamic link libraries (DLLs), complete executable programs and the like.

Common forms of computer-readable media may include, for example, magnetic disks, flash memory, RAM, a PROM, an EPROM, a FLASH-EPROM, or any other suitable memory chip or medium from which a computer or processor can read.

While the present disclosure describes various embodiments for illustrative purposes, such description is not intended to be limited to such embodiments. On the contrary, the applicant's teachings described and illustrated herein encompass various alternatives, modifications, and equivalents, without departing from the embodiments, the general scope of which is defined in the appended claims. Information as herein shown and described in detail is fully capable of attaining the above-described object of the present disclosure, the presently preferred embodiment of the present disclosure, and is, thus, representative of the subject matter which is broadly contemplated by the present disclosure.

What is claimed is:

1. A system for visualizing and managing health information data, the system comprising:
one or more subsystems adapted to receive health information data of one or more patients, wherein the one or more subsystems comprise one or more medical devices, electronic health records and inputs of a user;
a display device configured to display a viewing framework;
a computing system communicatively coupled to the one or more subsystems, the display device, a memory, and a processor, wherein the processor comprises program instructions that, when executed, causes the processor to perform operations comprising:
receiving the health information data from the one or more subsystems;
determining clinical context information of the health information data to generate contextualized data;
converting the contextualized data into usable data using trained machine learning models;
detecting viewing parameters, wherein the viewing parameters comprises one or more of: a display device type, a user's credentials, and a location of the display device;
determining the viewing framework based on the usable data and viewing parameters; and
sending the viewing framework to the display device automatically.

2. The system of claim 1, wherein the health information data comprises one or more of patient demographics, patient statuses, physiological measurements, medical history, medications, clinical notes, billing data, hospital guidelines and clinical research literature.

3. The system of claim 1, wherein the processor further performs operations comprising: determining one or more risk assessments related to the one or more patients; sorting the one or more risk assessments based on a level of risk; and identifying an alert relating to changes in the one or more risk assessments.

4. The system of claim 1, wherein the viewing framework comprises at least a graphical representation of one or more patient parameters relating to: vitals, medications, radiology tests, pre-operative data, intra-operative data, post-operative data, and fluid data.

5. The system of claim 1, wherein the viewing framework further comprises one or more risk assessments and one or more alerts.

6. The system of claim 5, wherein the one or more risk assessments is based on one or more thresholds relating to a condition of the one or more patients.

7. The system of claim 1, wherein the viewing framework further comprises a report displaying data tailored to one or more of a patient subgroup, a healthcare professional, and a hospital department.

7. The system of claim 1, wherein the viewing framework further comprises displaying one or more prompts for measuring vitals and taking tests, wherein the one or more prompts are updated and displayed based on a condition of the one or more patients.

8. The system of claim 1, wherein the viewing parameters may further comprise an index surgery, operative details, custom thresholds and a circle of care.

9. The system of claim 8, wherein the viewing parameters are customized by the user.

10. A method for visualizing and managing health information data, the method comprising:
receiving, at one or more subsystems, health information data of one or more patients, wherein the one or more subsystems comprise one or more medical devices, electronic health records and user inputs;
receiving, at a processor, the health information data from the one or more subsystems;
determining, at the processor, clinical context information of the health information data to generate contextualized data;
converting, at the processor, the contextualized data into usable data using trained machine learning models;
detecting, at the processor, viewing parameters, wherein the viewing parameters comprises one or more of: a display device type, a user's credentials, and a location of the display device;
determining, at the processor, the viewing framework based on the usable data and viewing parameters;
sending, at the processor, the viewing framework to the display device automatically; and
displaying, at the display device, the viewing framework.

11. The method of claim 10, wherein the health information data comprises one or more of patient demographics, patient statuses, physiological measurements, medical history, medications, clinical notes, billing data, hospital guidelines and clinical research literature.

12. The method of claim 10, further comprising: determining, at a processor, one or more risk assessments related to the one or more patients; sorting, at the processor, the one or more risk assessments based on a level of risk; and identifying, at the processor, an alert relating to changes in the one or more risk assessments.

13. The method of claim 10, wherein the viewing framework comprises at least a graphical representation of one or more patient parameters relating to: vitals, medications, radiology tests, pre-operative data, intra-operative data, post-operative data, and fluid data.

14. The method of claim 10, wherein the viewing framework further comprises one or more risk assessments and one or more alerts.

15. The method of claim 14, wherein the one or more risk assessments is based on one or more thresholds relating to a condition of the one or more patients.

16. The method of claim 10, wherein the viewing framework further comprises a report displaying data tailored to one or more of a patient subgroup, a healthcare professional, and a hospital department.

17. The method of claim 10, wherein the viewing framework further comprises displaying one or more prompts for measuring vitals and taking tests, wherein the one or more prompts are updated and displayed based on a condition of the one or more patients.

18. The method of claim 10, wherein the viewing parameters may further comprise an index surgery, operative details, custom thresholds and a circle of care.

19. The method of claim 18, wherein the viewing parameters are customized by the user.

20. A non-transitory computer readable medium storing program instructions, that when executed, cause a processor to perform operations comprising:
receiving the health information data from the one or more subsystems;
determining clinical context information of the health information data to generate contextualized data;
converting the contextualized data into usable data using trained machine learning models;
detecting viewing parameters, wherein the viewing parameters comprises one or more of: a display device type, a user's credentials, and a location of the display device;
determining the viewing framework based on the usable data and viewing parameters; and
sending the viewing framework to the display device automatically.
